(19) United States
(12) Patent Application Publication
(10) Pub. No.: US 2026/0151082 A1
(43) Pub. Date: Jun. 4, 2026

(54) SENSOR-ENABLED PERSONALIZED CARE AND WELLNESS MONITORING SYSTEM

(71) Applicant: LogicMark, Inc., Louisville, KY (US)

(72) Inventors: Chia-Lin Simmons, Moraga, CA (US); Rafael Saavedra, Cupertino, CA (US); Peter Williams, Belmont, CA (US)

(21) Appl. No.: 19/409,571

(22) Filed: Dec. 4, 2025

Related U.S. Application Data
(60) Provisional application No. 63/727,795, filed on Dec. 4, 2024.

(51) Int. Cl. A61B 5/00

(52) U.S. Cl. CPC A61B 5/4839; A61B 5/6889; A61B 5/7275; A61B 5/7465

(57) ABSTRACT

A person under monitoring (PUM) in a sensor-enabled environment (SEE) is monitored by a plurality of sensors that produce monitoring data. Based on the monitoring data, an agent is deployed or reconfigured. The deployed or reconfigured agent is configured to carry out various actions, which may include activating or reconfiguring further agents, and engaging with, prompting, or otherwise assisting the PUM.

### PRIORITY APPLICATION

This application claims priority to U.S. Provisional Application 63/727,795, filed Dec. 4, 2024, titled, “SENSOR-ENABLED PERSONALIZED CARE AND WELLNESS MONITORING SYSTEM”. The entire contents of said provisional application are incorporated by reference in the present application.

### BACKGROUND

The relationship between a person under monitoring and the systems that undertake monitoring of the person and their environment can be crucial to the role and effectiveness of that monitoring. The need for monitoring is often caused by well-being, health or safety concerns or issues of a person under monitoring (PUM). Monitoring can take many forms from, for example smart watches providing step counts or exercise goals, fall monitors, motion sensors, specialist devices intended for professional medical use.

Multiple types of sensors, and devices or systems communicating with such sensors may form a Sensor-Enabled Environment (SEE). The SEE may be situated in various locations, for example a location where a Person Under Monitoring (PUM) is predominately domiciled, for example a room, house or other domicile of a PUM. The sensors, devices or systems may be embedded in the environment, and can also include one or more worn or carried by the PUM or others s. A SEE can also include one or more systems for processing or managing the data sets generated by the one or more sensors, devices or systems present in the environment.

Applicant has filed various applications addressing different aspects of managing a sensor-enabled environment. These applications include: U.S. patent application Ser. No. 19/296,073, AUTOMATED ANOMALY DETECTION AND RESPONSE GENERATION IN A SENSOR-ENABLED ENVIRONMENT, filed Aug. 11, 2025; U.S. patent application Ser. No. 18/511,726, CARE VILLAGE DIGITAL TWIN SYSTEM AND METHOD, filed Nov. 16, 2023; U.S. patent application Ser. No. 18/104,117, ENVIRONMENT SENSING FOR CARE SYSTEMS, filed Jan. 31, 2023; U.S. patent application Ser. No. 18/919,216, GAME THEORY TOKEN MISALIGNMENT DETECTION AND REMEDIATION DEVICE, filed Oct. 17, 2024; U.S. patent application Ser. No. 19/088,709, LOCALIZED MACHINE LEARNING FOR MONITORING WITH DATA PRIVACY, filed Mar. 24, 2025; U.S. Patent Application Ser. No. 19/089,555, MACHINE LEARNING FOR AGGREGATING AND EVALUATING DATA FROM A SENSOR ENABLED ENVIRONMENT, filed Mar. 25, 2025; U.S. patent application Ser. No. 19/247,428, PERSONALIZED PHYSICS ENGINE, filed Jun. 24, 2025; U.S. patent application Ser. No. 18/129,713, SIGNAL PROCESSING FOR CARE PROVISION, filed Mar. 31, 2023; and U.S. Patent Application Ser. No. 17/972,389, SYSTEM AND METHOD FOR FALL DETECTION USING MULTIPLE SENSORS, INCLUDING BAROMETRIC OR ATMOSPHERIC PRESSURE SENSORS, file Oct. 24, 2022.

Generative AI, as its name suggests, is focused on the creation of new content, such as text, images, or code, based on input prompts. The large language model (LLM) is often used in generative AI, although there are other types of generative AI systems. For example, one can generate or edit content, and even perform simple function calling and chain together various options with an LLM. Agentic AI is a subset of generative AI that is centered around the orchestration and execution of “agents”, also termed AI agents, that use LLMs or other generative AI systems as a “brain” to perform actions through tools. Agentic AI goes beyond content creation and function calling by executing actions in underlying systems to achieve higher-level goals.

### SUMMARY

In one aspect of the present disclosure an example method of operating a sensor-enabled care environment is disclosed. The example method includes monitoring a person under monitoring (PUM) in a sensor-enabled environment (SEE) with plurality of sensors that produce monitoring data; and based on the monitoring data, automatically deploying or reconfiguring an agent, wherein the deployed or reconfigured agent is configured to carry out at least one action. The action the agent may be configured to carry out include: prompt the PUM to reduce risk to the PUM, identify an intended action of the PUM, assist the PUM in implementing the PUM's intended action, a ate a sensor in the SEE, reconfigure a sensor in the SEE, monitor the output of a sensor in the SEE, determine a state of the environment, determine a state of the, activate a second agent, and reconfigure a second agent.

In one alternative, the example method may include, based on the monitoring data, determining a state of the PUM has changed to a new state; based on the new state, selecting a type of agent to be deployed; and deploying the agent having the type selected. Alternatively, the example method may include, based on the monitoring data, determining a state of the environment has changed to a new state; based on the new state, selecting a type of agent to be deployed; and deploying the agent having the type selected. In another alternative, the example method may include, recognizing a pattern in the monitoring data; based on the pattern that is recognized, selecting the type of agent to be deployed; and deploying the agent having the selected type. Alternatively, based on the pattern that is recognized, an agent that is to be reconfigured is selected, and the new configuration of the agent that is to be reconfigured.

Optionally, in any of the above methods, the launched or reconfigured agent using an AI/ML system, may conduct a conversation with the PUM. Alternatively, the launched or reconfigured agent may be configured to at least one of: identify an intended action for the PUM, analyzing historical behavior data for the PUM with an AI/ML system to identify the intended action of the PUM, analyze historical behavior data for a population of similar individuals with an AI/ML system to identify the intended action of the PUM, analyze data patterns derived from the monitoring data to identify the intended action of the PUM, or analyze a model of the PUM as a physical object to identify the intended action of the PUM.

Optionally, the launched or reconfigured agent may be generate, with an AI/ML system, a prompt based on the intended action of the PUM; and prompt the PUM with the prompt to reduce risk to the PUM.

Optionally, the launched or reconfigured agent, may assist the PUM in implementing the PUM's intended action by at least one of notifying, by deployed or reconfigured agent, a caregiver to provide assistance, changing the state of the environment by the deployed or reconfigured agent by activating or changing the state of a device in the environment, or providing, by the deployed or reconfigured agent, information to the PUM.

In the example methods above may also optionally include (a) activating, by the launched or reconfigured agent, a sensor in the SEE or reconfiguring a sensor in the SEE, (b) monitoring, by the deployed or reconfigured agent, the output of a sensor in the SEE, (c) determining, by the deployed or reconfigured agent, a state of the PUM, (d) determining, by the deployed or reconfigured agent, a state of the environment, (e) activating, by the deployed or reconfigured agent, a second agent.

In all of the above methods, the agents may be AI/ML agents, for example using an LLM.

In other aspect of the present disclosure, an article of manufacture is disclosed. The article of manufacture may include a non-transient computer readable medium having stored thereon instructions configured, when executed by a processor, to cause the processor to carry out any of the above example methods.

In another aspect of the present disclosure, an example system is disclosed. The example system may include a plurality of sensors configured to monitor a PUM in a SEE and produce monitoring data; a data repository configured to store historical data regarding the PUM; a processor in communication with the plurality of sensors and the data repository and configured, based on the monitoring data, to deploy or reconfigure a plurality of agents, each of the plurality of agents configured to at least one action selected from the group consisting of: provide an interactive engagement with the PUM, prompt the PUM to reduce risk to the PUM, identify an intended action of the PUM, assist the PUM in implementing the PUM's intended action activate a sensor in the SEE reconfigure a sensor in the SEE monitor the output of a sensor in the SEE, determine a state of the environment, determine a state of the PUM, and reconfigure or activate a second agent.

In one alternative, the example system may include storage accessible to the processor, storing at least one of the monitoring data or patterns derived from the monitoring data, the deployed agent having access to the monitoring data or patterns. In another alternative, the example system may include an AI/ML system configured to identify patterns in the monitoring data, the processor configured to select the type of agent to deploy or reconfigure based on the identified patterns. In another alternative each of the agents further comprise a respective AI/ML model. The AI/ML system may be configured to identify patterns in the monitoring data, and the agent's respective AI/ML models configured to receive the identified patterns and carry out the action based on the identified patterns.

### DETAILED DESCRIPTION OF THE DRAWINGS

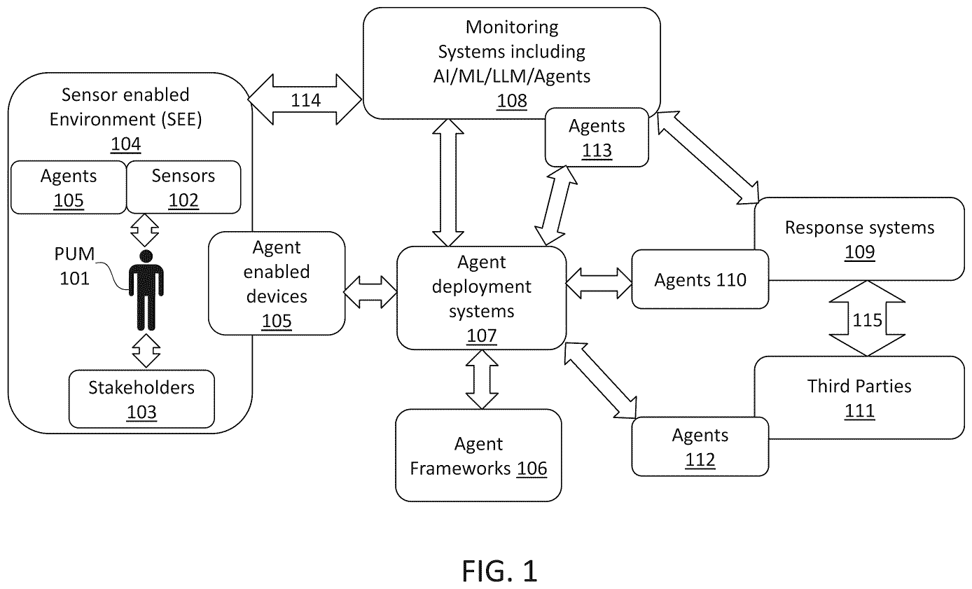

FIG. 1 illustrates an example sensor-enabled monitoring system that includes one or more AI/ML systems and agents, according to an example embodiment of the present disclosure.

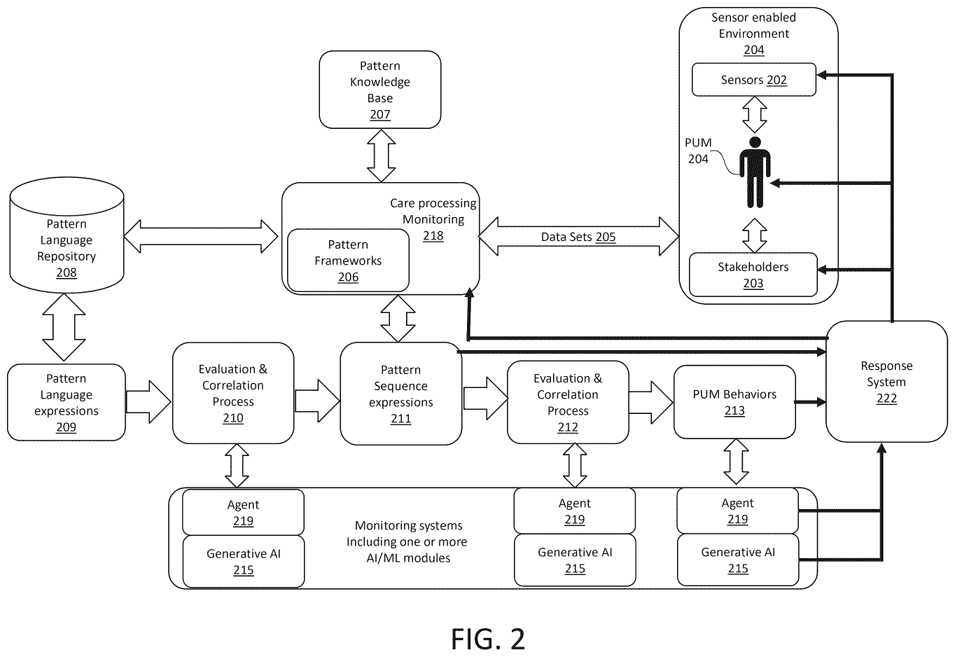

FIG. 2 illustrates the use of a pattern language in an example monitoring and response system deployed with an SEE, according to an example embodiment of the present disclosure.

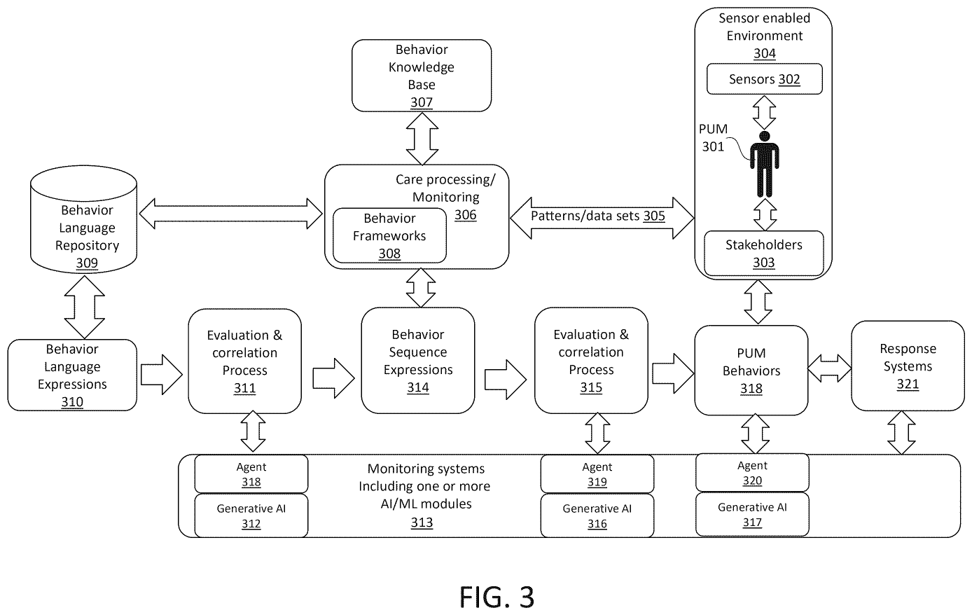

FIG. 3 illustrates use of a behavior language in an example monitoring and response system deployed with an SEE, according to an example embodiment of the present disclosure.

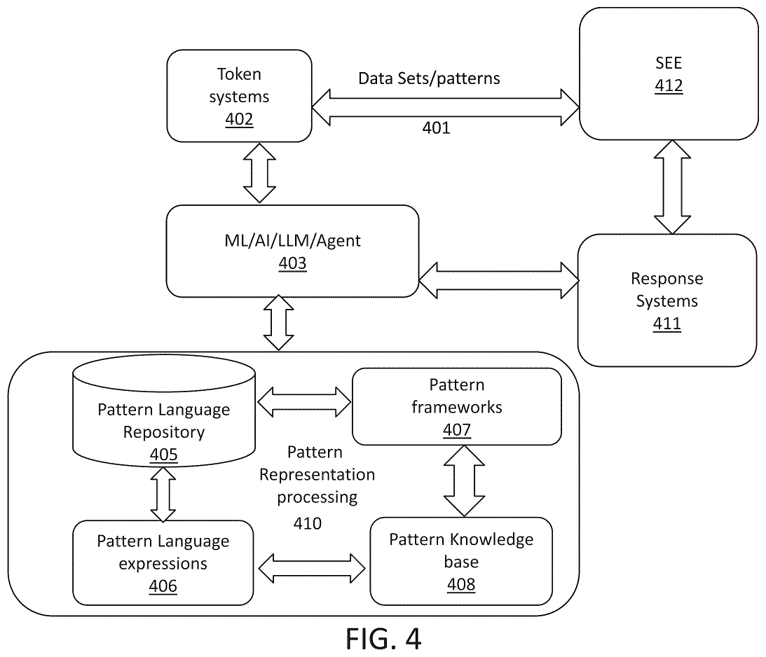

FIG. 4 Illustrates use of a pattern language with an AI/ML system in connection with an example SEE and response system, according to an example embodiment of the present disclosure.

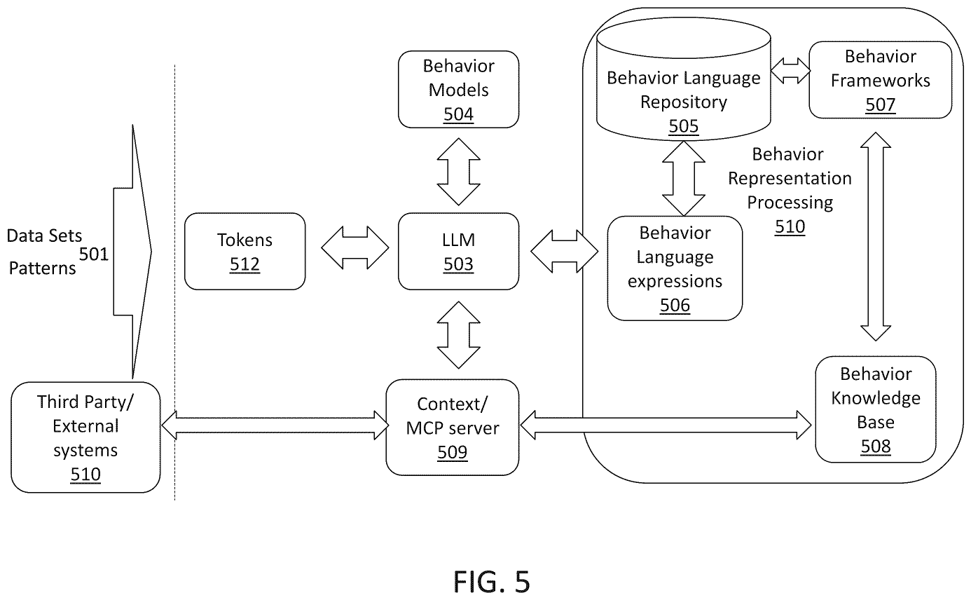

FIG. 5 illustrates use of a behavior language with an LLM, according to an example embodiment of the present disclosure.

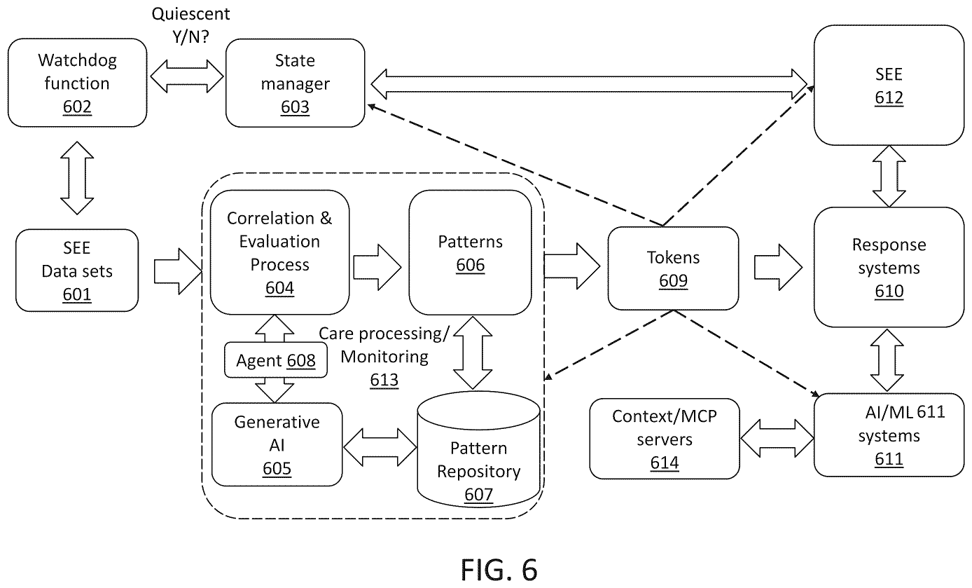

FIG. 6 an example system which identifies state of data sets generated in connection with an SEE, according to an example embodiment of the present disclosure.

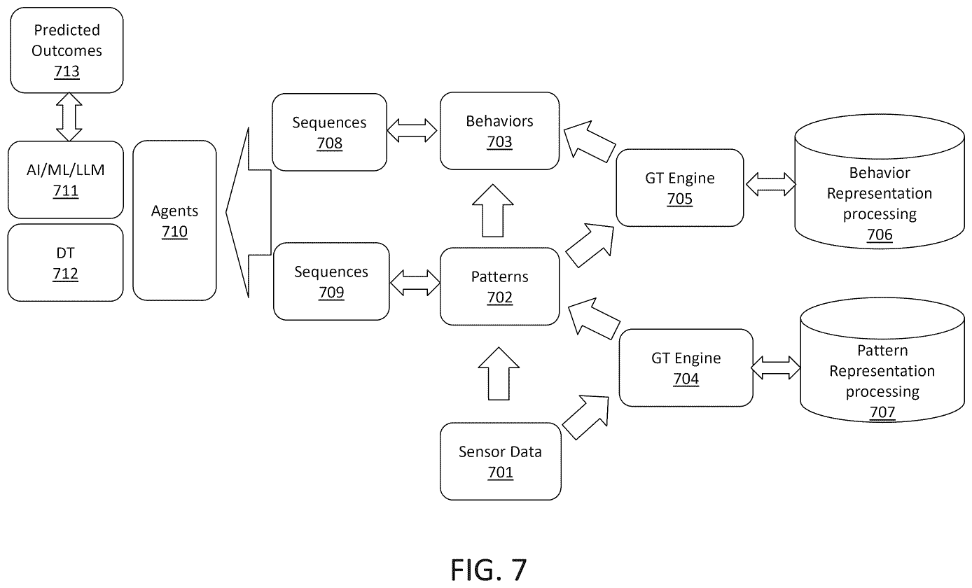

FIG. 7 illustrates an example game theory engine, according to an example embodiment of the present disclosure.

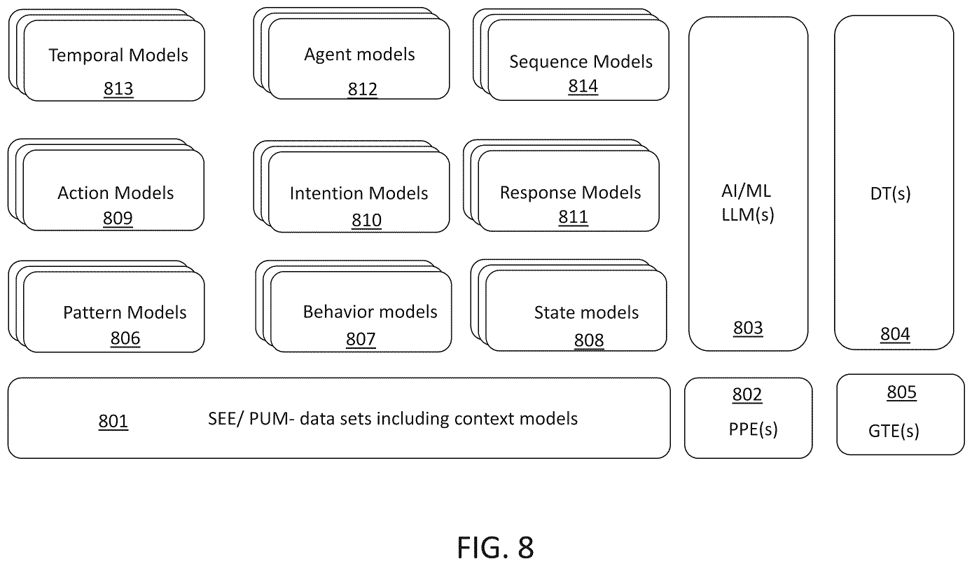

FIG. 8 illustrates models in an example SEE monitoring system, according to an example embodiment of the present disclosure

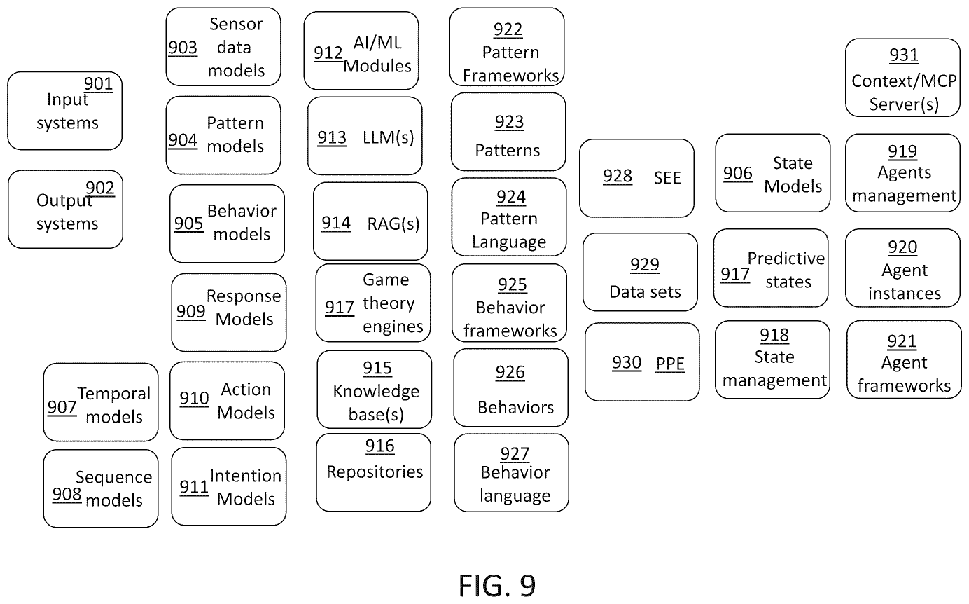

FIG. 9 illustrates an example AI/ML-based monitoring a system, according to an example embodiment of the present disclosure. The example system

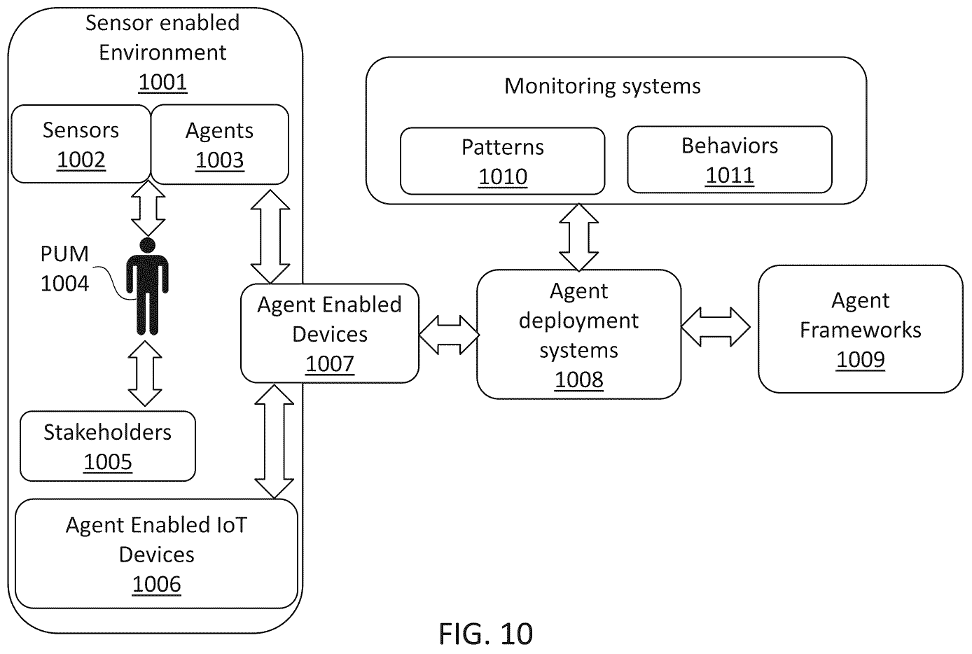

FIG. 10 illustrates agent deployment and configuration, according to an example embodiment of the present disclosure.

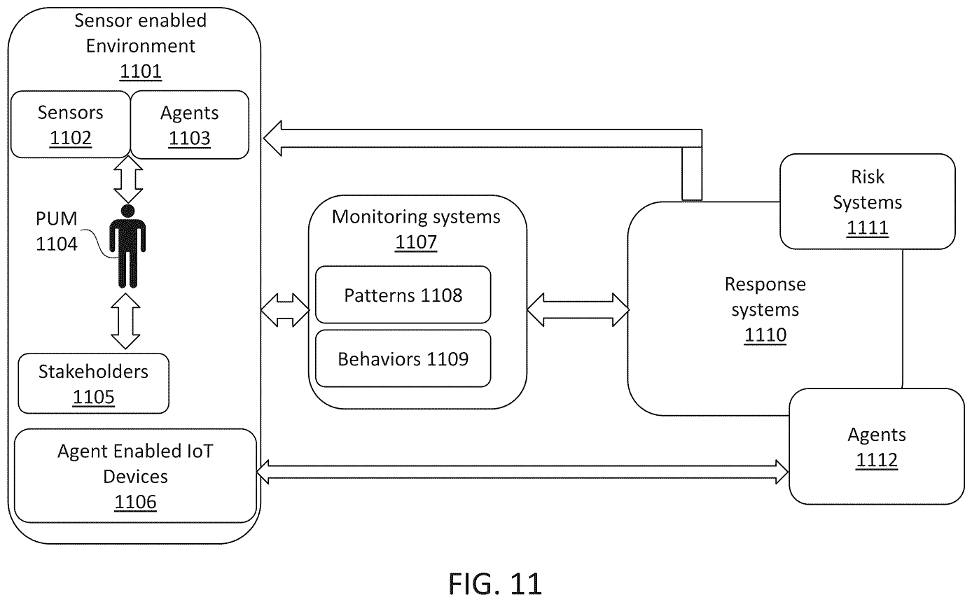

FIG. 11 illustrates monitoring and response system used with a sensor-enabled environment (SEE), according to an example embodiment of the present disclosure.

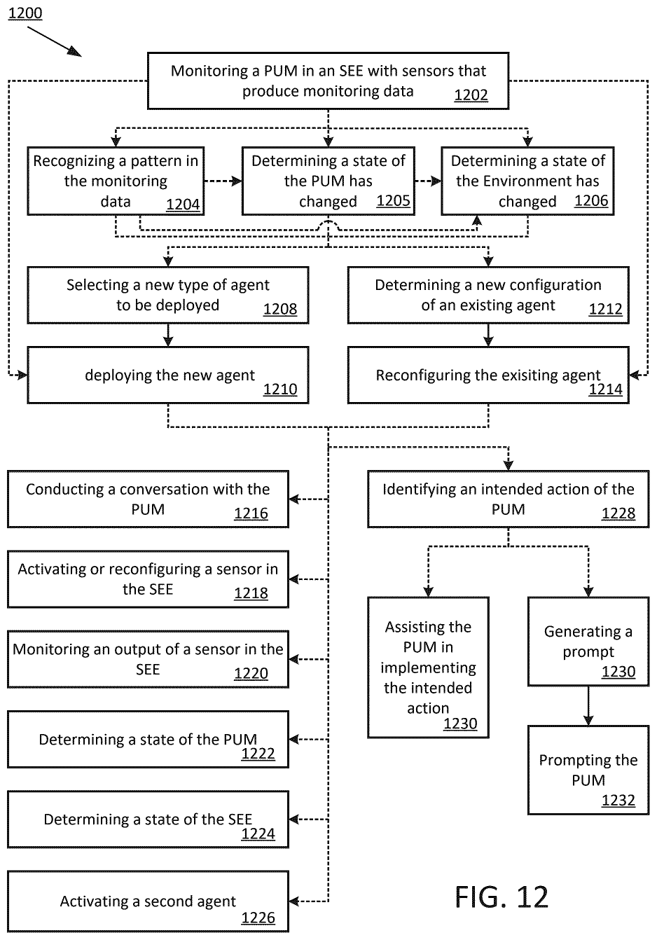

FIG. 12 illustrates a flow diagram of an example method, according to an example embodiment of the present disclosure.

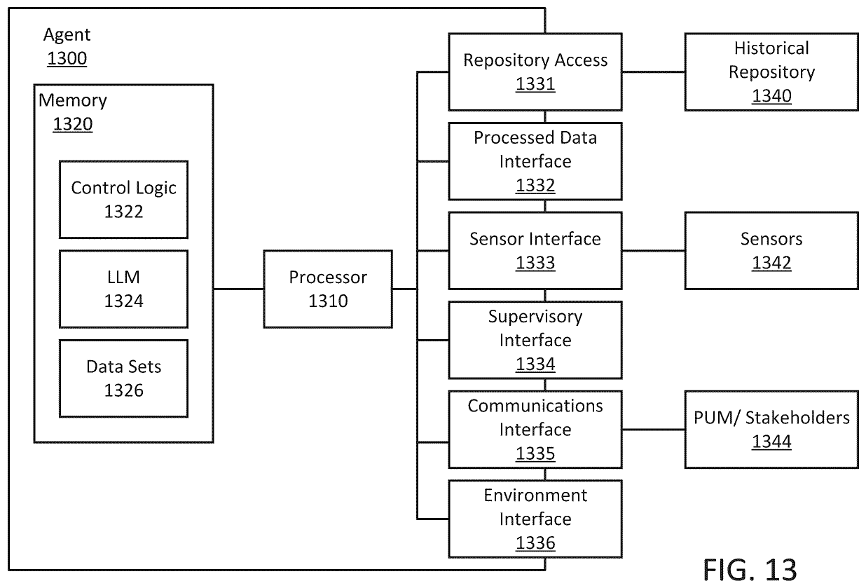

FIG. 13 illustrates an architecture for an example agent, according to an example embodiment of the present disclosure.

### DETAILED DESCRIPTION OF EXAMPLE EMBODIMENTS

In some embodiments, a SEE can operate as a single entity with multiple sensing capabilities. Multiple sensing capabilities including, e.g., visual, RADAR, LIDAR, audio, haptic, locational or temporal sensors can be used to identify the behavior of the person being monitored. The SEE may also replicate the monitoring of a PUM within the calibration, configuration and operations of the sensing capabilities of the one or more sensors, devices or systems present in the SEE, using, for example, one or more digital twins.

How the SEE supports the wellbeing, care, health or safety of the PUM may, at least in part, determined by the relationship the PUM has with the SEE. The degree of trust the PUM has in the SEE and in any data, messages, responses, actions or impacts that are direct or indirect results from the SEE operations, including measurements, or interactions between the SEE and PUM may impact the operation of the SEE and its effectiveness in supporting the PUM's care, health, or safety.

Such a relationship can be enhanced by protection of the privacy of the PUM. Privacy may be enhanced if not all the data that is generated by the capabilities of the one or more sensors, devices or systems present in a SEE is be made available to one or more monitoring systems or other stakeholders involved in the monitoring operations. For example, a camera may be used to generate edges or vertices from an observed area that includes a PUM, however the images from which such edge or vertices were generated might not be made available to any monitoring system unless there is a detrimental health or wellness event for a PUM, such as a fall, heart attack, stroke or the like. When such an event occurs, such images may then be made available to an EMT, Carer or other authenticated health professional. Privacy may also be enhanced using the systems and methods of U.S. patent application Ser. No. 19/088,709, LOCALIZED MACHINE LEARNING FOR MONITORING WITH DATA PRIVACY, filed Mar. 24, 2025.

The use of one or more AI/ML systems within the SEE or elements thereof, can further enhance or exacerbate relationship attributes between the PUM and the SEE, e.g., the perceptions of the PUM such as trust in, or reliance on the See or other. Such a relationship may be enhanced by improving the reliability of the AI/ML system operations such that they do not generate false or unreliable conclusions, which can have a detrimental impact on the PUM, or on the PUM's perception of the system.

The embodiments described herein may include data sets generated by one or more sensors, devices or systems. The data sets can be arranged into one or more configurations. For example, one configuration can be in form sequences or patterns, which can then form or represent behaviors, in whole or in part. This particular arrangement is well suited to the use of AI/ML systems, such as LLMs or other generative AI systems. The groupings and arrangements of data can form a language that is parsed by the AI/ML system. In some embodiments, such languages may comprise information from sets of data generated by the one or more sensors, devices or systems, where such data, for example forms a stream that is then segmented or quantized, including by the use of metrics or tokens, into one or more formats that are suitable for one or more AI/ML system to operate upon.

The structure, hierarchy or arrangement of these data sets can be configured to facilitate the use of more AI/ML systems. Such arrangements or configurations may be made to optimize such data sets to improve the effectiveness, accuracy or reliability of such AI/ML systems.

The deployment of ML and AI capabilities can provide substantial benefits to a system for monitoring the care, wellbeing, health or safety of a person under monitoring (PUM), including the identification of current or potential patterns or behaviors, including events that can have an impact on the PUM or other stakeholders. These capabilities can facilitate the monitoring of those variations that occur over extended periods of time, such as a gradual decrease in mental acuity, reduction in mobility, increase in weight or other gradual changes to be identified prior to any potential detrimental occurrences that can be the result of these deteriorations, such as a fall, stroke, heart attack and the like.

The use of AI/ML for the measurement, monitoring or prediction based on data sets generated by the one or more sensors, devices or systems present in a SEE can include one or more generative AI systems such as LLM, including LLMs that are personalized to a specific person, location or group, that evaluates human behaviors represented as a language describing, at least in part, the human condition in the context of a person being monitored in a sensor-enabled environment.

FIG. 1 illustrates an example sensor-enabled monitoring system that includes one or more AI/ML systems, according to an example embodiment of the present disclosure. The AI/ML systems may include generative AI systems such as LLMs, or agents. A PUM (101) may be domiciled or present in a SEE (104) where there are one or more embedded, worn or carried sensors, devices or systems (102) which can have one or more agents (105) bound to them or operating with them, and one or more other stakeholders (103). The SEE can generate one or more data sets. The data sets can be represented as patterns or behaviors (114) that can form inputs to one or more monitoring system (108) which can include one or more agents (113). These inputs, can be processed or operated upon, in whole or in part, by one or more processing systems, including care hubs or care processing systems. Such data sets, patterns or behaviors may be stored in a manner which is accessible, in whole or in part within such monitoring systems (108). Such monitoring systems may include one or more AI/ML systems, generative AI systems such as LLMs. The monitoring systems can process such inputs and generate one or more communications, which can be provided to one or more output systems. The output system can include, for example risk management, impact analysis, interaction, response or other systems (109). These systems may include, or have associated with them, agents (110), e.g., AI/ML system based agents. These outputs of the output systems (109) may form, in whole or in part, communications to the one or more PUM, Stakeholders, Sensors, devices, systems, or agents, such as agents running on agent-supported devices (105) present in a SEE. The output can include, for example, calibrations, configurations, interactions, and other communications. In some cases, these outputs can be human or machine interpretable. These outputs can, in some embodiments be communicated to one or more agents, including agent-supported devices (105), including those worn or carried by a PUM or other stakeholder. Further outputs (115) can be communicated to third parties (111), for example service providers, financial or insurance institutions or other product or service providers, where for example one or more agents (112) can also be deployed. In some embodiments, the deployment or configuration of such agents can be undertaken by agent deployment systems (107), which can use agent frameworks (106).

Alignment involves the arrangement of one or more systems entities in a relationship with other entities so as to optimize the performance of each entity individually and in combination with other entities with which there is a relationship with that is aligned.

### Patterns

In some embodiments, data sets generated by the one or more sensors, devices or systems involved in the monitoring of an environment, for example a SEE, can form patterns, in whole or in part. These patterns may include representations of the activities, events or state of that environment, including any PUM or other stakeholders present therein.

These patterns can represent sets of data that are generated by the one or more sensors, devices or systems present in a SEE. These data sets can, in some embodiments, be organized into quantized sequences or elements, for example to represent a movement, activity, event or other state of the SEE or the stakeholders therein. Tokens may be used to represent a pattern. In some embodiments, this can include recognition of a word, phrase or sound generated by a PUM. One or more sensors may configured for such recognition or pattern identification or matching, e.g., using their own associated agent, where one or more pattern characteristics are matched to those previously identified or stored.

In some embodiments, a token can be any format, from simply being a representation of a set of data to a more structured representation comprising multiple segments which can have one or more meta data bound to them, by reference or embedding. A token that has a cryptographic formation that may be configured such that the data the token represents is only available to authorized, authenticated persons, for example recognized stakeholders, or entities, including one or more sensors, devices or systems present in a SEE. For example, public key encryption may be used to create tokens that require a private key to access the data represented therein. Tokens may be cryptographically signed or encrypted depending on the intended use of these tokens. The data sets represented by the tokens may range from simple integer/characters/algorithms to complex or organized sets of data that can form ontologies, hierarchies, taxonomies or other organizational data structures, which in turn may have at least one set of specifications governing, in whole or in part, their representation, communication, storage, disclosure or other characteristics.

For example, the data set of a pattern can form the data represented by a token, which may be, for example cryptographically signed and can include the one or more sources of the data, the timing of such data generation, one or more locations of the sources, one or more configurations of the sources at the time or location of the data generation or one or more specifications representing the relationships of the sources.

Such patterns of generated data can be quantized or classified by the one or more sensors, devices or systems generating the data, by one or more systems such as care hub, care processing or by one or more AI/ML system, including generative AI systems such as LLMs, or agents, the operations of which can include one or more game theory systems.

Some of the sensors, devices or system or system or server arrangements in some SEE embodiments, can be configured to include machine learning or statistical mechanisms as adaptive methods to identify patterns that indicate changes in the state of the user or the environment to more accurately or dynamically select sensor configurations or trigger events, notifications or responses. These machine learning techniques can include one or more agents trained on models of such data that include patterns that have been identified, including by one or more AI/ML system, including for example neural networks, and can include use in whole or in part of those models. For example, an agent may be trained on a model that includes identified patterns for a PUM in a SEE in a period of time, for example a week or month, where the categorization of the patterns as represented by the model, includes, for example, patterns that are correlated to a specific location, time, activity, behavior, state or other recognizable artifact of the model. For example the agent may be trained only on that part of the model that is correlated to a PUM location, for example the kitchen of the environment, a PUM activity, for example walking, sleeping and the like, one or more PUM health aspect, such as breathing, coughing or similar, which can be part of the HCP (Health Care Profile) of the PUM or one or more states of the PUM, including the environment, for example where that state transitions from quiescent. For example, data sets transmitted by the sensors in a PERS device or from the one or more sensors, devices or systems of the SEE, can be stored and used by one or more servers for training machine learning systems or to supply CVDT (Care Village Digital Twins) representing a user and their environment, supporting adaptations to changes in the user's behaviors or in the environment. The digital twins may be based on the systems and methods disclosed in U.S. patent application Ser. No. 18/511,726, CARE VILLAGE DIGITAL TWIN SYSTEM AND METHOD, filed Nov. 16, 2023. This approach may increase accuracy of predictions, to better anticipate and prepare any response, including those of an agent. The agent may obtain emergency or other support resources, and/or provide notification of a carer, in response to changes in the detected patterns.

In some embodiments, an agent can be configured to communicate with the PUM or their carer's providing each with notifications that are pertinent to the state of the PUM, for example represented by one or more patterns or behaviors, where these patterns or behaviors indicate a health, wellness or safety issue for the PUM. For example the communications with the PUM, though one or more devices present in the SEE, including those worn or carried by the PUM, may advise the PUM to cease an activity, sit or lie down, change their location and the like, all of which may be presented in a manner and style that is reassuring to the PUM, whereas the communications to the carer may include notifications as to the severity of the issue and can include remedial actions such as medications, alerts to emergency services, provision of wheel chairs or other assistance.

The agent configuration can include multiple potential responses which are evaluated by the agent based on the training data and the data sets generated by the one or more sensors, devices or systems of the SEE and can include the use of digital twins, where for example an agent my instantiate or configure one or more operating digital twin, including any predictive or generative systems thereof, for example one or more AI/ML systems or agents, with data sets that comprise the patterns recognized by the agent and data sets provided by the SEE, to evaluate potential outcomes.

In some embodiments risk metrics can be bound through reference or embedding, for example as attributes, parameters, variables or other characteristics, to one or more identified elements of an environment, for example a set of stairs can have a higher risk metric than a flat surface or a flat surface that is wet can have a higher risk metric than one that is dry. Such risk metrics can be defined as the inherent risk metrics of an environment, which can be incorporated into one or more risk calculations, for example when a PUM is present or interacting with those environments.

In some embodiments, risk metrics may be calculated, at least in part, by monitoring data sets generated by the one or more sensors, devices or systems present in the SEE. For example, worn or carried devices may measure, for example heart rate, sweat, eye movement or other physiological characteristics and the measurements may be correlated to the PUM current state. The risk associated with a state may be predetermined, or adjusted based on additional information. For example, walking down stairs can have a risk metric of (X), and if the stairs are in darkness, then the risk metric may be higher (2X).

In some embodiments each pattern or combinations thereof forming a behavior representing a PUM, for example sitting, standing, walking, sleeping, eating and the like can have one or more risk metrics bound to them. For example, sleeping may have a lower risk metric than running or walking.

One aspect of risk identification is balancing the potential risk with any observed or measured risk. For example, if a PUM trips over a carpet or pet, there are a number of potential outcomes each with a risk of that outcome occurring. For example, if the PUM falls, this has a number of possible outcomes, ranging from serious outcomes, such as broken hip, head injury or another life threatening situation, to minor outcomes, such as the PUM regaining their balance and possibly stubbing their toe.

The identification of the risks is, at least in part, determined by the measured data from the one or more sensors, devices or systems present in the SEE. In some embodiments, one or more sensors may detect an edge condition, e.g., one that changes the quiescent state to a more active or dynamic state. For example,, an initial change in orientation of the PUM, such as when they trip over an object may be detected by, for example an accelerometer detecting an increase in acceleration of the upper body, a visual sensor capturing the vertical orientation of the PUM or an audio and/or haptic sensor detecting a noise or impact. These sensors can provide this data to one or more risk identification systems. The risk identification systems can include one or more agents configured to evaluate these data sets and generate or predict one or more risk metrics for this occurrence.

A risk identification systems can be configured to evaluate data sets provided by, for example, one or more sensors, devices or systems of the SEE or their proxies. For example, agents may be configured to evaluate individual or aggregated data sets of those sensors, devices or systems in order to generate a set of risk or safety metrics that correspond to the risks of a set of outcomes based on the current or predicted data set from the one or more sensors, devices or systems. For example, the one or more sensors, devices or system present in the SEE detecting, hesitation of a PUM in a particular behavior, which may indicate the PUM's concern for safety.

In some embodiments a PUM may exhibit a set of behaviors, which are measured by the one or more sensors, devices or systems of the SEE. These behaviors may be represented by one or more patterns. For example, a PUM may exhibit the following activities which can be represented by a set of patterns that represent a behavior. In this example a PUM is initially sitting, then attempts to rise to stand, however they grasp at a wall or other support, for example furniture, and achieve a state of standing, though still holding onto the furniture or wall for support. In this example, there can be a sequence of patterns, which comprise the date sets generated by the one or more sensors, devices or systems of the SEE that correspond to these activities. For example, pattern 1 (P1) represents the PUM sitting, pattern 2 (P2) represents the PUM rising from sitting to standing, pattern 3 (P3) represents the PUM reaching or grasping for support as they undertake P2 and pattern 4 (P4) represents the PUM standing, albeit with support from furniture, a wall or similar.

Each of these patterns can have states. For example, if there is no measurements to the contrary, P1 can be a quiescent state. However, if there are measurements of breathing, blood pressure, heart rate or other data sets, for example generated by a worn device, then the state of this pattern can be non-quiescent and can include one or more risk metric representing the potential for the measured state to have a health, wellness or safety impact.

In the example of P2, the quiescent state of that pattern is when the PUM successfully rises from sitting to standing with no outside assistance or within the thresholds of their normal movements. Consequently, risk metrics for P2 in this state can be below any thresholds for notifications or actions. However, in this example, one or more devices in the SEE, for example mm wave radar, camera, worn device and the like may have detected that the alignment of the PUM body does not conform with their normal execution of this pattern. For example, the camera may be configured to observe the vertical alignment of the PUM body, for example using edge detection and potentially using a personal physics engine (PPE) configured for that PUM, such as the engine described in U.S. patent application Ser. No. 19/247,428, PERSONALIZED PHYSICS ENGINE, filed Jun. 24, 2025. Further detection can include acceleration measurements, for example from a worn device, which can include three dimensional measurements and time. In this example, P3 is recognized as the PUM reaching or grasping for support, and as such the state of P2 is changed from quiescent, representing the normal activity to a state indicating the difficulty the PUM is experiencing. The state change of P2 can include changes in the risk metrics of this pattern from below any threshold, to heightened metrics representing potential issues for the PUM, such as falling, injuring themselves in other ways and the like. In differing embodiments, the granularity of these risk metrics can range from simple binary to complex algorithms.

As P3 has been identified, the state of P3 can, for example, be quiescent, for example where the PUM is using a hand rail to steady or guide themselves, to another state, which in this example includes risk metrics that exceed one or more thresholds for that PUM. These risk metrics can include parameters such as location, time or other contextual characteristics. As the PUM finally achieves a standing position, represented by pattern (P4), the state of this pattern can include recognition that they are unsteady in that condition and as such the risk metrics of P4 can represent this state.

The sequence of patterns, P1 through P4 can represent a behavior, rising from sitting, that has been undertaken by the PUM on many previous occasions, where the state of such behavior can be quiescent, in that there is no elevated risk to the PUM, to a failed state where for example the PUM is unable to stand. In this example, where the PUM has achieved a standing position, albeit with some degree of difficulty (P3), the state of the behavior can include the aggregation of the patterns and the one or more risk metrics of those patterns to provide an overall risk metric for the behavior. For example, once standing (P4) the PUM may undertake a further activity, such as walking to another location, for example the kitchen, which can be represented, for example by pattern 5(P5 ). In the example of them successfully undertaking this activity, the risk metrics may be lowered and the state of the PUM for that pattern may revert to being quiescent. However, if they continue to reach for support (P3) then the risk metrics and state may be varied accordingly. In some embodiments, P1 through P5 may form a behavior (B1), for example couch to kitchen which has been previously measured by the one or more sensors, devices or systems of the SEE.

In some embodiments one or more agents may be deployed or configured based on the state or risk metrics of the one or more patterns or behaviors of the PUM. For example, an agent (A1) may be operating in the device being worn by a PUM and configured to measure, oxygen flow, acceleration, skin temperature or breathing using the capabilities of the device. This agent may also be communicating with Agent2 (A2) which is configured to evaluate data sets generated by one or more sensors, devices or systems of the SEE, for example a camera that is configured to capture the edges of the PUM body, an audio sensor configured to recognize breathing patterns of the PUM and a mm radar that measures the position of the PUM in relation to one or more identified locations, for example a couch.

In some embodiments both Agents (A1) and (A2) may be configured to monitor the state of the PUM through correlation with the patterns of the PUM, in that if the PUM state varies from quiescent, for example one or more pattern (P1, P2, P3, P4) varies, then the agent may generate a notification or action based on this change in state.

Many current monitoring systems that include alert and notification functionality operate on a binary basis, that is in an example of a threshold based system either the data is above or below threshold. This is limiting and has a high risk of generating false positives as well as missing leading indicators that can be used to predict future health, wellness or safety issues.

The use of patterns or behaviors that can represent data sets generated by the one or more sensors, devices or systems of a SEE for monitoring a PUM, coupled with the awareness, by one or more monitoring systems, of state of the environment and the PUM therein and the use of one or more risk metrics can provide one or more AI/ML system including agents with sufficient data of an appropriate granularity to support care of the PUM, through optimization of the measurements, increased efficiently of the processing of such data and higher resolution of the outcomes, including predictions of future health, wellness and safety issues.

The deployment and configuration of one or more agents configured to evaluate differing sets of data representing the activity of a PUM in a SEE may facilitate a significantly higher resolution of understanding of the PUM condition and the context thereof. For example, where the state of the PUM and environment is quiescent and based on predictions, for example using one or more AI/ML systems, digital twins and PPE, has a high probability of remaining as such, there may be a limited deployment of agents that, for example are configured to monitor behaviors and sets thereof, for example sleeping, waking, eating and the like.

The deployment of other agents, for example those configured to evaluate specific conditions, for example coughing, breathing difficulties and the like, specific locations, for example bedroom, bathroom or kitchen or those configured to evaluate specific patterns, (for example P1, P2, P3, P4) or behaviors (for example B1) can, in some embodiments, be triggered by, for example, a change in state (actual or predicted), increase in risk metrics, a specific pattern (for example pattern (P3), mentioned herein) and can be initiated by, for example, an overall monitoring system or by one or more agents or AI/ML systems configured to do so.

In some embodiments, agents can be configured to initiate communications with one or more stakeholders, including the PUM, when, for example, one or more criteria matching the configuration of the agent is met. These communications can include interactions with the one or more stakeholders and can include a first agent initiating the deployment and configuration of a second agent that, for example, is configured to interact with a stakeholder, for example a PUM, where the interactions are determined, at least in part, by the context of the PUM. For example, if a PUM has fallen, then the agent may, using one or more devices present in a SEE capable of communication with the PUM, for example smart speaker, smart TV, or other capable IoT devices, worn or carried devices, including PERS devices, smart watches, smart phones, earbuds and the like, engage the PUM in a conversation or interaction. This can include establishing the ability of the PUM to communicate, that can include evaluating whether they are they conscious, and capable of communication. For example, depending on the response or not of the PUM, the agent may directly or indirectly, for example though a monitoring system or another agent, communicate the state of the PUM, including any data sets relevant to their current health state, to one or more other stakeholders, for example a carer or emergency services.

If the PUM is capable of communicating with an agent, then the agent can interact with the PUM through, for example a query/response interaction, to establish the health state of the PUM. For example, if the PUM has fallen and is capable of the communications and interactions, the agent may lead the PUM through a self-diagnosis process to establish, at least in part, the state of the PUM and to vary the risk metrics based on that information. This can include informing, for example a carer or other stakeholder of the situation and if the risk metrics are, for example, within a set of tolerances, that is between one or more thresholds, then for example emergency services may not be contacted immediately.

In this example the communications and interactions with the PUM may include deployment and configuration of a further agent which has been configured with a personalized style of communications that is empathetic to the PUM, for example offering reassurance using, for example a sympathetic tone whilst engaging with the PUM to establish, at least in part, the health condition of the PUM. This can include the agent representing this condition in the form, for example of a set of risk metrics, for example ranked by highest risk to life to lower risks. For example, if the PUM has a heart condition, then the highest risk may be a heart attack and as such the agent may communicate directly or indirectly with the one or more sensors, devices or systems of the SEE to measure the state of the PUM in regard of that condition. For example, observing the movements of the PUM to detect any contractions of the chest, breathing irregularities and the like.

In this example a set of agents can be deployed and configured, where one of these agents may act to triage the operations of these agents and provide access to further resources, such as PPE, digital twins or one or more AI/ML systems. For example, the communications among and between the agents may be modulated by this or another specifically configured agent to support communications to and among the one or more sensors, devices or systems of the SEE are not contradictory and include a priority, for example based on the risk metrics.

The use of digital twins in this situation can facilitate the one or more agents predicting the potential impact of any response, especially by the PUM to this current situation. For example, if there is a risk metric indicating potential further impairment if the PUM undertakes a particular movement, then this may be communicated to the PUM or other stakeholders including the carer. In this example, specialist medical personnel may be alerted to this prediction and can be dispatched to the location of the PUM or further supporting activities, such as emergency surgery or other time critical activities at can be scheduled at accessible medical locations, for example a hospital. In this example an agent may interact with the PUM to reassure them that assistance is on the way and help them in remaining conscious, whilst monitoring any indications of alarm or panic that could have detrimental impact on their health condition.

Depending, at least in part, on the risk metrics of the agents and monitoring systems of the SEE or including the communications or interactions with the PUM, the responses of the agents can include escalating or deescalating those responses.

In some embodiments an AI/ML system can be trained on sets of data generated by the one or more sensors, devices or systems in a SEE. This training can include the topographical mapping of these data sets based on their location, overlap of the data sets and the one or more events, occurrences or actions, including change of state that has initiated a potential pattern. This topological mapping can include the use of sparse mapping where significantly less data is employed in the training process, however that data includes context, such as timing, locational, relationship of the source to other data sources, state of the environment and the like.

In some embodiments these data sets can be evaluated as a field, similar to the evaluation of a magnetic field, where each of the individual data points is represented in the context of the other data points forming such a field.

For example, each sensor can have a spatial relationship with those other sensors forming the SEE, such that as each data set generated includes spatial and temporal characteristics which are retained. In this manner these data sets can be stored locally or remotely, however only certain data may be passed to an AI/ML for evaluation. For example, this can include data that exceeds or is within one or more thresholds, which can form part of the configuration of the one or more sensors, devices or systems. These data points can form a sparse topological representation of the SEE or the PUM therein.

This can include data sets that have been previously generated or those generated in real time.

This approach can reduce the amount and time of any training and increase the efficiency of that processing, consequently reducing the resources required.

Each of these patterns, having been identified, classified or bound to one or more identifiers can form a set of tokenized elements that can, in some embodiments form sequences, where specific patterns can form sequences that are repeated. For example, a sequence may comprise Pattern A, Pattern B, Pattern C and Pattern N, where each these patterns are sequential and generated by sensors, devices or systems present in a SEE. For example, in some embodiments, Pattern A may be generated by Sensor A, Pattern B by device B, pattern C by system C and pattern N by a combination of one or more sensors, devices or systems, identified as sensor set Q. In this example each of the generated patterns can be generated at the same time or in the same time interval.

In some embodiments, a single pattern may be represented in multiple tokens, each of which can have differing characteristics. For example, a token that is communicated to an AI/ML system, including one that uses a digital twin may differ from that received by a care signal processing system. Tokens may be constructed, for example, to have multiple layers or segments, where for example the data sets comprising the pattern, for example those from differing sensors, in each layer or segment may represent differing amounts of detail, fidelity, historical, contextual, temporal data or the like.

A further example may involve the same patterns generated by one or more sensors in a specific time period of duration (X), where the sequence of the patterns A, B, C and N is consistent over that period.

These sets or sequences of patterns can form the basis of a language of patterns, where such quantized patterns can represent the words thereof. These words can form phrases where a specific sequence can represent, for example the movements of a PUM within a SEE, for example moving from a sitting position to standing or vice versa.

The use of one or more AI/ML systems, including LLMs that have been trained on the data sets generated by the one or more sensors, devices or sensors of the SEE, to recognize and manage the patterns represented by such data, can generate and operate on such pattern language.

In some embodiments, patterns can be represented by tokens, where for example a data set from one or more sensor, device or system that is determined to represent a pattern can have a unique identifier and form part of one or more organizations or arrangements of such patterns.

In some embodiments, a token may comprise a set of segments, each of which can have a differing access method. Such multi segment tokens can include binary or a text string uniquely referencing a data set, which can be all or part of a pattern, by reference or embedding. The configuration of a token and one or more end points, for example one or more agents, can include configuration so as that only specific end points may be able to access specified segments of a multi segment token, for example in a token with, for example, there are 5 segments, different end points may only be able to access, for example segment 3 or 5 of that token.

In some embodiments, one or more game theory games can be deployed to determine which sensor's data sets can form, in whole or in part, which patterns. For example, if a pattern has N data sets for Y sensors, devices or systems, and such pattern can be incomplete or the probability of the data sets forming such pattern is below a threshold, one or more games may be invoked such that the sensors, devices or systems generated data sets become the moves or steps within the game where the sensors, devices or systems are players and the payoff is the reliable identification of a pattern.

Games can also be employed to, at least in part, determine the likely set of patterns that may occur, based on, for example, sparse data sets, including for example those that can form parts of multiple patterns. This can include, for example, those data sets that have relationships with other data sets, for example those simultaneously generated, sequentially generated, triggered by an event or action, forming part of a timed duration and the like. In this example, such data sets may have relationships with each other, for example using one or more organizing approach, including for example, identifiers, arrangements, dependencies or other organizations, such as ontologies, taxonomies, hierarchies or other arrangements.

In this manner there may be networks of data sets, that can for example, form part of one or more game strategies, in that, for example if data set X is recognized, then a sensor, device or system acting as a player in a game may contribute data set Y, which for example was generated in the same time interval for the same environment, as both data sets can form part of a pattern (G). One aspect of this approach is the determination of the minimal initial data set that indicates one or more patterns.

In some embodiments, the state of a PUM, other stakeholder, SEE or elements thereof, can have state, in that for a specific time period, including sets and sequences thereof, the state may be represented by a pattern, for example a quiescent state pattern. Similar to human language communications, these quiescent state patterns can be considered as periods of silence or non-communication, in that the state of the patterns is such that there is no representation or communication of any data that affects the PUM, other stakeholders or the SEE. In some embodiments one or more AI/ML systems, including agents may evaluate such pattern data to identify any indication of the change of state of that pattern, where such data is not sufficient to trigger any threshold or other specifications that determine the state of the pattern and yet may indicate, through for example variations in one or more sensor, device or system data set, that there is a trend towards a change in state, which can include vectors. For example, this can include the use of digital twins where such AI/ML systems, one or more game theory games or other techniques are employed to extrapolate these variations to identify, at least in part, the potential for a state change, which can be represented as a probability, vector or other metric.

For any one or more sensors in a SEE, there can be a quiescent state, from the perspective of a system monitoring the environment, where the sensor is either not providing any data to the system or there is no change in that data. Sensors can have state, in that they are operating and at least one of collecting, measuring, processing, storing or transmitting data to the systems that have configured the sensor and established the command and control of the sensor operations.

In an environment with multiple sensors, each of these states of each sensor may in part be configured as part of a system to establish a quiescent state for a monitored environment comprising a number of sensors. This can include one or more sensors, measuring and sensing the environment. For example, there may be no transmission of data to other devices or systems from a sensor or set thereof, based on the state of the sensor and the environment, individually or collectively. This can include the sensor being inactive and operationally dormant or the sensors being configured to only transmit data on an action, event, trigger, threshold or occurrence either generated externally, for example by the system, or derived from the sensor measuring or processing capabilities. The combination of states of individual sensors may be integrated such that a care analytics management processor, or other monitoring system, including for example one or more AI/ML systems, which can incorporate one or more sets of command and control options, can configure their operational state or manage which sensors may communicate with other sensors to vary operating states.

One aspect is the calibration of the SEE, establishing a quiescent state of an environment, such that the “at rest” state of the SEE as whole may be used, at least in part, in any evaluations of any activities, changes, or variations within that SEE, including those of a PUM or other stakeholder. Establishing the quiescent state of the SEE may support identification of any variances from that state. In some example embodiments one or more test procedures of one or more sensors, devices or systems, including arrangements thereof, may be instigated as part of the calibration process. These test procedures may include, for example, active or passive elements such as audio generators, impact generators or color balance displays. For example, a calibrated and initialized sensor, device or system set forming the calibrated and initialized SEE, which can include care hub, care processing and any other monitoring or management systems, the one or more physics engines, (including PPE) and any AI/ML systems, including agents, that can create a representation of the state of the SEE, where each sensor, device or system set measures, at least in part, the SEE in a quiescent state. These measurements, can represent the state of the SEE and the one or more sensors, devices or systems therein, in any arrangement. Establishing the state where the measured activity, if any, of the monitored environment, including the PUM or any other stakeholder therein, is evaluated as being quiescent, that is “at rest” where there is nothing occurring that has any health, wellness or safety impact or risk for the PUM or other stakeholders. This is the quiescent state of the SEE and the one or more sensors, devices or systems therein. This can include those times without a PUM or other stakeholders being present and can be either contiguous or segmented, covering 24-hour clock time.

In some embodiments a state manager can be employed to, for example, represent the state of the SEE and the one or more sensors, devices or systems therein, represented for example as measurements that are stored in one or more repository. This repository can be distributed, for example where each sensor, device or system stores their state, for example at or over a period of time, including the quiescent state. The state manager can have multiple representations of the state data, for example the state as stored by the one or more sensors, devices or systems, care processing systems, monitoring systems or one or more separate repositories of the state manager. In this manner the data sets representing state can be compared to, for example, reference data sets to ensure that the sensor, device or system state data is correct. In some embodiments, three or more data sets may be maintained to ensure that the state of the SEE is accurate.

In some embodiments, a state manager can incorporate or have access to one or more repositories that include thresholds, triggers, vectors or other representations, including for example patterns, or behaviors, that represent a change of state of the one or more sensors, devices or systems of the SEE in any arrangement.

In some embodiments, a sensor, for example a haptic, temperature, audio, humidity or other sensor may generate a set of data that is expressed as a set of integers. In many cases these integers may have little or no variance over time unless and until an event occurs. For example, a temperature sensor that is operating correctly generates data that matches the variations in temperature in a SEE, where such variation is generally incremental. In this example the slope or vector of the variation is that part of the data that can be informing, and as such collecting and tokenizing each measurement provides minimal benefit to one or more monitoring systems or AI/ML systems. In this example, an agent may be configured to monitor such temperature sensor and only provide an output when the measurements indicate a change in state that indicates a risk to the PUM.

In some embodiments, such variations, represented for example by vectors, may form part of a quiescent state, where such state is defined by one or more thresholds representing the degree of variation and the timing of such variations, including the vectors and slopes thereof. This data may be tokenized and used, for example, by one or more AI/ML system including an LLM to model these variations.

In some embodiments one or more Personal Physics Engines (PPE) may be operating where data sets from the one or more sensors, devices or systems, which can include patterns representing those data sets, are correlated, at least in part, with the movements of a PUM, which can also be represented by patterns, managed by the PPE.

In some embodiments, patterns which have a state that is not quiescent, which can be represented by a state token, where such state is determined, at least in part, by such pattern meeting one or more criteria, such as exceeding a threshold, matching a vector, or matching an event or action, or may include one or more data sets that are generated by the one or more sensors, devices or systems present in a SEE and can include further configurations of such sensors, devices or systems including the data sets generated by such configurations.

In some embodiments, a pattern framework can comprise or more data sets or patterns that can represent a pattern of data that represents a recurrent activity of a PUM or other stakeholder and can generally, in some embodiments form part of a behavior. For example, reaching to grab an object, clenching of the fists, smoothing the hair and the like can all be patterns that can be identified by the one or more sensors, devices or systems present in a SEE, including those worn or carried by a PUM or other stakeholders. In some embodiments the sequence of patterns can be indicative or a behavior or activity by the PUM and such sequence can be used to identify where the state of the PUM may have a health, wellness or safety risk or impact. These pattern frameworks can be identified, for example, using the one or more sensors, devices or systems of a SEE and can include the use of a PPE, AI/ML, agents and the like. When a pattern framework has been identified, it can be managed by, for example a care processing or monitoring system.

In some embodiments a pattern knowledge base can comprise a repository of all the identified patterns for a PUM or other stakeholders and can include, for example patterns of one or more other PUM or other stakeholders which have similar conditions, situations or other shared characteristics. These may have been identified in other SEE involving different PUM or stakeholders, and have been anonymized.

In some embodiments, a pattern language repository may comprise a set of language elements that can represent one or more patterns of a PUM or other stakeholder. For example, this can include syntax and semantics, and can comprise, for example, verbs and nouns, where the nouns are identified pattens, for example those stored in a pattern knowledge base.

FIG. 2 illustrates the use of a pattern language in an example SEE, according to an example embodiment of the present disclosure. A PUM (101) is present in a SEE (204) that includes one or more embedded, carried or worn sensors, devices or systems (202) and can include one or more other stakeholders (203). The SEE (204) generates one or more data sets (205), which can be communicated to one or more care processing systems or monitoring (218), including for example care hubs, which can include one or more pattern frameworks (206). These data sets (205) may then be processed by the care processing systems (218), using for example pattern knowledge base (207), pattern frameworks (206) or pattern language repository (208) in any arrangement to determine the pattern representing the current state of the SEE, including the PUM. A care processing or monitoring systems can include one or more game theory engine, AI/ML systems, including agents, or other general or specialized modules for the processing of the data sets to identify, generate or represent, in whole in part one or more pattern language expressions (209). These expressions (209) may then be evaluated or correlated (210), which can include an generative AI system (215) such as an LLM or an agent (219) employing one or more models, which can be represent one or more pattern sequence expressions (211), which can include the output of the generative AI system or agent, for example as predictions of the next or following patterns in a sequence that represents the state, activity or events of the SEE, including those of the PUM. Such sequence expressions (211) may undergo evaluation or correlation processing, including interacting with an appropriately configured generative AI system (216) or agents (220) including the models thereof to represent one or more PUM, or other stakeholder, behaviors (213), which can include the state, activities or events occurring in a SEE, including those involving the PUM. These behaviors can be communicated to one or more response system (222), to an appropriately configured generative AI system (217), such as an LLM, or agents (221) and the models thereof. The generative AI systems or agents can generate one or more responses, and can be communicated through a response system, including for example as a pass through, for example as a token that can be accessed by a specific entity, for example sensors (202), stakeholders (203), such as a carer, or the PUM (204). The response system, can provide responses to, for example, sensors (202), for example as configuration variations, Stakeholders (203), for example through for example a response system to the SEE (204), including to the PUM (201), stakeholders (203), for example as alerts, a PUM (204), for example as alerts or health, wellness or safety communications directly to them or to their carried or worn devices, and to the monitoring systems. The response systems (222) may process or act upon multiple data sources, for example in the form of pattern sequence expressions (211), PUM behaviors (213), LLM (217) and/or agent (221) outputs in any arrangement. The monitoring systems (214) incorporating the one or more LLM (215, 216, 217) and agents (219,220,221) can form part of a care process and monitoring system (219) or can operate as a separate system.

### Pattern Language

A pattern language can include one or more data sets or sequences thereof generated by the one or more sensors, devices or systems in a SEE, where these are recognized as patterns by one or more monitoring systems, including AI/ML systems. These patterns can be persisted or represented as one or more tokens.

In some embodiments, a pattern language can include a set of tokens that represent one or more data sets of one or more sensors, devices or systems, where such tokens can include:
Action tokens (verbs)
State tokens (nouns)
Context tokens (locations/temporal/ . . . )
Threshold tokens (degree/metrics/ . . . )
Movement tokens (sitting/rising/walking . . . )
Condition tokens (falls/breathing difficulties/ . . . )
Special tokens (Start of pattern/end of pattern/type of pattern)

These tokens can be arranged to represent data sets generated by the one or more sensors, devices or systems, so as to convey, at least in part, the patterns representing the state of the environment and any stakeholders present therein. For example, this could include a token designated “stand” which represents the one or more patterns of a PUM as they rise from sitting to standing, a token designated “walk” representing a PUM walking, which may be from the sofa (being location 1), to the kitchen (being location 2).

In some embodiments a vocabulary of patterns may be generated, based on the observed and identified patterns of one or more stakeholder. For example, when a set of sensors, devices or systems data sets representing the state of the SEE, include data that is representative of a stakeholder undertaking an action. For example, one or more haptic sensors, for example mounted on the floor of a SEE, may detect footfall of a stakeholder, which for example, may be matched to that of a PUM, providing, at least in part, a pattern of movement of that PUM such as them walking from one location to another. One aspect of this approach is determining the possible patterns, such as footfall, hand, arm or other limb movements, grasping, for example a cup or other object, using hands or fingers to select or control an object, patting or other hand/arm movements or the like.

In some embodiments, one or more game theory games can be employed to, at least in part, determine with a minimal amount of data possible the set of possible patterns that, based on this initial data set are most likely to occur. This use of sparse data sets can include one or more AI/ML techniques, such as principal components analysis, hashing, dimension reduction or entropy weighted algorithms and the like. These techniques may be combined with game theory games, where the strategies of the games are aligned to the possible outcomes, which may be based on aggregated data sets of similar situations, previous verified and validated patterns or behaviors or other known outcomes, such as those held in a knowledge base of data sets or patterns or those predicted by one or more AI/ML systems. Such an approach can include, for example each of the one or more sensors, devices or systems employing one or more AI/ML systems or agents or one or more configurations to, at least in part, identify one or more minimum data set for pattern selection. This can include identification or selection of a minimum data set for identification of an entry point to one or more pattern/behavior organizational structure(s), for example a knowledge base or vocabulary or other repository.

FIG. 7 illustrates identification of patterns using a game theory engine, according to an example embodiment of the present disclosure. Sensor data (701) may be evaluated or processed by a game theory engine (704) which can include the deployment and use of one or more games to, at least in part identify one or more patterns (702). This can include the game theory engine identifying specific games or game strategies that can optimize this identification. The game theory engine and the games deployed by same, can then interact with pattern representation processing (707), to further identify, manage or store the patterns (702) and data sets (701) thereof. Similarly, patterns (702) can form, in whole or in part, one or more behaviors (703), and can interact with game theory engines (705) that are configured to facilitate one or more games to be deployed to identify the one or more behaviors (703). This can include interactions with behavior representation processing (706). Bothe the patterns (702) and behaviors (703), where either or both can form sequences (708,709). These patterns (702), behaviors (703) and the sequences thereof (708,709) can be communicated to one or more agent, including multi agent systems (710), which can represent one or more AI/ML systems, including LLMs (711) which can use one or more digital twins (712) and produce one or more predicted outcomes (713) in any arrangement.

In some embodiments, patterns may include one or more special tokens that can be recognized by one or more AI/ML systems, including one or more agents, for example an LLM. Such special tokens can form part of the pattern itself, for example pattern start/pattern end and or may represent the pattern in whole, for example pattern (N), for example PUM rises from location 1 and walks to location 2, or can represent the state of the pattern, for example quiescent or may represent other characteristics or meta data of the pattern any arrangement.

In some embodiments, an LLM may be employed to generate one or more patterns, based at least in part on the data sets or patterns that are provided to such an LLM by the one more sensors, devices or systems, including agents, present in a SEE. These data sets or patterns may be specific to a SEE or one or more stakeholders therein, including a PUM.

The LLM, may be used, at least in part, to evaluate, for example, one or more sequence of patterns that can equate to a sentence, paragraph, conversation, potentially involving multiple stakeholders, locations, SEEs or other parties.

FIG. 4 Illustrates use of a pattern language with an AI/ML system in an example SEE, according to an example embodiment of the present disclosure. The AI/ML system may include an LLM or agent. One or more data sets or patterns (401) generated by the one or more sensors, devices or systems of the SEE (412) can be converted into one or more tokens (402) where such tokens are suitable for evaluation or processing by an AI/ML, including LLM or agents (403). This AI/ML (403) can interact with one or more pattern processing systems (410). The pattern processing systems 4100 may include one or more pattern language repository (405), one or more pattern language expressions (406), for example in the form of a vocabulary and one more pattern knowledge bases (408), which can interoperate in any arrangement. Each of the pattern representation processing entities, singly and in any combination can interact with the AI/ML, including LLM or agents (403). The outputs of the AI/ML (403) can be communicated to one or more response systems that can instigate one or more response, for example communicating with a SEE (412), including the sensors, devices or systems therein, the stakeholders thereof, including a PUM in any arrangement, These communications can include calibrations or configurations of the one or more sensors, devices or systems or communications, for example alerts, notifications and the like to the one or more devices worn or carried by one or more stakeholder, including the PUM.

### Behaviors

In some embodiments, one or more patterns may form, in whole or in part a behavior of a stakeholder. For example, a behavior may be making tea or coffee or other beverage, making and then eating lunch, sleeping, reading, walking, watching TV, playing a game and the like. Each of these behaviors can include a set of patterns, which in turn may include data sets generated by the one or more sensors, devices or systems present in an environment, for example a SEE.

In some embodiments a behavior can be represented by one or more token, where for example, a single behavior may be represented in multiple tokens, each of which can have differing characteristics.

This approach can facilitate one or more systems, including those employing one or more AI/ML systems, including LLMs, to anticipate or predict the likely behaviors of a stakeholder, for example a PUM. This can include detection of changes in that behavior, for example if the PUM's health or wellness is deteriorating or improving. The identification and detection of such deterioration or improvement can involve understanding the behaviors of a PUM over a period of time, such that the natural rhythm of their behaviors provides, for example, the training data for one or more AI/ML system, such that if and when those behaviors vary, the identification and detection of such variance can be undertaken in advance of any potential impact of that variation.

For example, if a PUM exhibits a small but increasing variation in their behavior, for example a limp or other observable physical variation, such as an increase in shaking of their hand, the systems using the AI/ML capabilities can detect and predict a set of likely outcomes that may represent a detrimental health and wellness impact, enabling the provision of appropriate remedial actions to avoid or mitigate such outcomes.

In some embodiments, the behaviors can form a language, where for example such language is composed of the “words” of the pattern language and the behaviors are the phrases and sentences. Such a behavior language can represent the behaviors of the stakeholders, including the PUM, and lead to a rich and detailed understanding of the dynamic inter day and day to day activities of multiple stakeholders, including their interactions.

This can include the monitoring of the sequences of the various behaviors, in that if there is a typical repeated sequence, such as waking, breakfast, bathroom, walk for the morning and then this sequence varies, for example waking, bathroom, breakfast, bathroom, walking, then such variance can indicate a change in that stakeholder's health or wellbeing. Such variance may be reported to one or more other stakeholders including a carer, or may be communicated to one or more AI/ML systems that has been configured to assist in the care of the stakeholder. For example, such an AI/ML system may, for example through a device controlled by the stakeholder, inquire as to any variance in the diet of the stakeholder or may suggest such a variance.

One aspect of behavior monitoring is the identification of the start, duration, conclusion or context of a behavior. For example, if a PUM rises from sitting to standing, there may be multiple behaviors that can start from this initial action. Such standing movement can, in some embodiments, be represented by a set of patterns, which in turn may be represented in the form of data sets, including those generated by a PPE (personal physics engine).

In some embodiments, such initial action can be represented by an AI/ML system, such as an LLM, where the initial behavior, in this example rising from sitting to standing, is evaluated in the context of the possible further behaviors that may be undertaken. For example, this could include traversing a location to, for example a bathroom, kitchen or bedroom, each of which may involve a differing behavior. These alternatives can be represented, for example, within a digital twin, where the PUM representation includes a PPE, and the context of the behavior, such as time, presence of other stakeholders, relationship to other behaviors or patterns, including sequences thereof, can be evaluated by the AI/ML systems to generate in one or more digital twins the probable behaviors of the PUM.

In this manner the determination of the intentions of the PUM and their degree of success in achieving that intention, through measurement of their activities by the SEE, can be in part or in whole be determined by such AI/ML systems, including LLMs, including using the one or more digital twins This can facilitate one or more AI/ML systems to, support or in other ways assist the PUM in achieving their intention, through configuration of the one or more sensors, devices or systems in the SEE and any variations to the environment that can be undertaken by the AI/ML systems.

In some embodiments, one or more behavior models may be instantiated by the one or more AI/ML systems, such as an LLM, including those involving digital twins. These models can include sets of behaviors for individual PUMs, representing their behaviors over various time periods. For example, a model for PUM (X) can include their behaviors for each day of the week, such that their regular, scheduled or usual behaviors are represented in one or more digital twins, which can be used in conjunction with the one or more sensors, devices or systems that are present or employed in monitoring that PUM to, at least in part, evaluate those behaviors so as to identify and health, wellness, care or safety risks or impacts that can affect the PUM.

Such AI/ML systems may provide reminders, assistance, messages, communications or trigger other actions or activities. This may include configurations or commands to one or more sensors, devices or systems. These AI/ML system-provided signals may be based at least in part on the models used for monitoring the PUM, recognized patterns or behaviors, or the data sets generated by the one or more sensors, devices or systems.

In some embodiments, behavior recognition can include sensor, device or system-facilitated pattern detection. The detected one or more patterns may form, at least in part, part of multiple behaviors. This pattern analysis can be based on video, audio, haptic, textual, locational, temporal, environmental or external data sets generated by the one or more sensors, devices or systems of the SEE.

One aspect of the determination of a PUM or other stakeholder behavior is the recognition of a transition state from one behavior to another. It may be helpful if quantization of such behaviors is sufficiently accurate. Sufficient accuracy may facilitate the one or more monitoring systems, including AI/ML systems or agents, such as an LLM with or without a RAG, to establish reliable and accurate correlations of the measured inputs from the one or more sensors, devices or systems. Accurate correlation may permit the recognition of the initiation of one or more behaviors various action or events, or conclusion of a specific behavior. The use of tokens, based for example on patterns, that can form behaviors which can be employed by one or more AI/ML systems, including LLMs, to predict which of a set of possible behaviors, based on one or more patterns, is the most likely to occur. This can inform the one or more monitoring systems, including for example care hub or care processing, such that they may undertake a risk evaluation, communicating with, for example AI/ML facilitated risk management systems. This can include the use of one or more digital twins as part of this evaluation. This evaluation can be communicated, in whole or in part to one more devices, including for example those worn or carried by a PUM, where the one or more risks may be communicated to the PUM, through for example an agent deployed on such devices or to other stakeholders, including caregivers, through their agent or other application devices.

In some embodiments a behavior, can be represented by a token, where that token includes an identifier or meta data that includes, at least in part, description of the behavior. Representation of behavior may also include a set of attributes, for example, those of a PUM including intention, attention, focus, outcome or those of the context of the behavior, for example temporal characteristics including absolute and relative timing, duration or whether the behavior is continuous or segmented or whether the behavior is scheduled, occurs at a regular time or is spontaneous. This meta data can include or link to further data sets, e.g., those declared or stated by the PUM for a behavior. Further attributes or meta data can include the relationship of a behavior to other behaviors, the sequencing of behaviors, as well as the relationship of one or more behaviors to other contextual elements, such as the presence or absence of other stakeholders, external events, such as weather or the like.

One aspect of this approach is the determination, at least in part, of the sequence of one or more behaviors. A behavior that is out of sequence can be identified and any risks of such behavior rapidly communicated to the relevant stakeholders, including for example the PUM, or to one or more sensors, devices or systems. Communications to sensors, devices, or systems, may alter their configurations, increasing the fidelity or efficiency of such monitoring.

In some embodiments one or more models, including those generated through the training of one or more AI/ML systems, including one or more LLMs, may include organizations of patterns and behavior relationships. These organizations can include ontologies, taxonomies, hierarchies or other arrangements that can be instantiated in the form, for example, of graph databases or other repositories. This can include one or more sets of weightings or other attributes, such as vectors, that can be used by one or more AI/ML system, such as an LLM, to predict the intended behavior of a PUM or other stakeholder with minimal pattern input. This may facilitate the early detection of such behaviors and the configuration of one or more sensors, devices or systems to support the PUM in the undertaking of such behavior leading to a favorable outcome for the PUM. For example, this can include providing prompts, risk assessments, advice, alerts or other communications that can assist the PUM or other stakeholders and may include variations to the operations of one or more sensors, devices or systems in the SEE, including for example one or more appliances or other environment systems capable of having their operations varied, for example an HVAC system, smart TV, smart fridge or other IoT systems.

In some embodiments, one or more transition patterns may be identified, where for example one behavior transitions to another. This can include identification of the sequence of such patterns or behaviors so that if behavior A is followed by behavior B, then transition pattern X can be detected before behavior A concludes. This has the advantage that any end point detection for the conclusion of a behavior does not have to be bound to strict or rigid specifications, but can include fuzzy or variable end point detection whilst maintaining accuracy.

In some embodiments, there may be a repository, for example a graph database that includes one or more patterns and their relationships to one or more behaviors. For example the repository may indicate an initial pattern or set of patterns that form part of a behavior. For example, pattern 1, for example a hand movement, can have relationships with multiple behaviors, such as holding a glass, cup or mug, grasping an object, banister, can or other furniture, signaling to another stakeholder, and the like. The sequence of patterns, for example if pattern 1 is followed by pattern 2, where pattern 2 is the closing of the hand on an object, may constrain the range of possible behaviors. These sequences of pattens that can form behaviors can inform the one or more care hubs or care processing systems as to the likely behavior of the stakeholder, and in some circumstances can provide, in whole or in part, indications as to the intent and consequent success of the stakeholder behavior.

Such a repository may be used, in some embodiments, to provide contextual data or RAG capability to one or more LLM which can be employed to predict the likely behavior of a stakeholder. This can include contextual data including data generated by the one or more sensors, devices or systems of the SEE and can be in the format of Model Context Protocol (MCP).

In some embodiments, a behavior framework can comprise or more data sets or patterns that can represent a behavior or a PUM or other stakeholder that can form part of a further behavior. For example, rising from sitting to standing may be a behavior that can form part of many other behaviors, usually those with further context, such as walking to the kitchen, bathroom or other location. These behavior frameworks can be identified, for example, using the one or more sensors, devices or systems of a SEE and can include the use of a PPE. When a behavior framework has been identified, it can be managed by, for example, a care processing or monitoring system.

In some embodiments a behavior knowledge base can comprise a repository of all the identified behaviors for a PUM or other stakeholders and can include, for example behaviors of a PUM or other stakeholder which have similar conditions, situations or other shared characteristics. These may have been identified in other SEE involving different PUM or stakeholders, and have been anonymized.

In some embodiments, a behavior language repository may comprise a set of language elements that can represent one or more behaviors of a PUM or other stakeholder. For example, this can include syntax and semantics, and can comprise, for example, verbs and nouns, where the nouns are the behaviors, for example those stored in a behavior knowledge base.

FIG. 3 illustrates use of a behavior language in an example SEE, according to an example embodiment of the present disclosure. The SEE (304), can include a PUM (301) or other stakeholders (303) and includes one or more embedded, worn or carried sensors, devices or systems (302) generating one or more data sets, which can include one or more patterns (305). The SEE (304) can generate one or more data sets (305), which can be communicated to one or more care processing systems (306), including for example care hubs or monitoring systems, which can include one or more behavior frameworks (308), for example as a repository. These data sets or patterns (305) may be processed by the care processing or monitoring systems (306), using for example behavior knowledge base (307), behavior frameworks (308) or behavior language repository (309) in any arrangement. Care processing or monitoring systems (306) can include one or more game theory engines, e.g., analogous to those described in U.S. patent application Ser. No. 18/919,216, GAME THEORY TOKEN MISALIGNMENT DETECTION AND REMEDIATION DEVICE, filed Oct. 17, 2024, AI/ML systems or agents or other general or specialized modules for the processing of the data sets or patterns to identify, generate or represent, in whole in part one or more behavior language expressions (310). These expressions (310) may then be evaluated or correlated (311), which can include an generative AI system such as an generative AI system such as an LLM (312) or agents (318) and one or more models thereof, to represent one or more behavior sequence expressions (314). Such expressions (314) may undergo evaluation or correlation, including interacting with an appropriately configured generative AI/LLM (316) or agents (319) and the models thereof to represent one or more PUM (301), or other stakeholder (303), behaviors (318). The monitoring systems (313) can interact with one or more response systems to communicate with the sensors (302), stakeholders (303), PUM (304) in any arrangement which can include one or more configuration variations, alerts or notifications including those communicated to the one or more devices carried or worn by the PUM (304) or stakeholders (303). In some embodiments, communications to and amongst the one or more LLMs (312,316,317) may be direct or may be through one or more agents (318,319,320), including multi agent configurations in any arrangement. The monitoring systems (313) incorporating the one or more LLM (312, 316, 317) and agents (318,319,320) can form part of a care process and monitoring system (306) or can operate as a separate system.

### Language of Behaviors

In some embodiments, behaviors can be represented by tokens where these tokenized behaviors can represent, in whole or in part the aggregated behaviors of a PUM over a period of time. These tokens can comprise one or more patterns, which may also be represented by tokens. The behavior and pattern tokens can comprise the data sets generated by the one or more sensors, devices or systems of a SEE that are employed to monitor and measure the state of that environment and the stakeholders therein, including the PUM. This can include the representation of sequences of patterns or behaviors, represented by tokens, that taken as an aggregate can, represent one or more human behaviors in the form of an individualized “behavior language” describing a human condition in the context of a PUM. This behavior language may be evaluated by one or more AI/ML systems, including LLMs, to identify or predict likely PUM patterns or behaviors.

Each of the behaviors can be constructed from one or more patterns in an arrangement, where these patterns in turn comprise one or more sets of data in an arrangement, where those sets of data are generated by the one or more sensors, devices or systems present in a SEE. In some situations a data set or pattern may form part of multiple behaviors, and in the case of the initial data set or pattern of a behavior, where for example data set 1 or pattern 1, is the initial element in a behavior, and these data sets or pattern can be the initial element in multiple behaviors, one or more AI/ML systems, including for example LLMs, may be invoked, for example using a digital twin, to predict or identify the behavior that is most likely to occur. This can be achieved by evaluation of the patterns representing the data sets or a behavior as a form of language, where the cadence, semantics or syntax of that language can include the movements, activities or utterances of the PUM or other stakeholders as they undertake their intended or actual behaviors.

As each of the data sets or patterns can be represented by a token, these tokens can be evaluated by an AI/ML system, for example an LLM/LCM, where the weightings, attention or vectors of the LLM/LCM can provide the capability of determining, at least in part, the behavior, including the sequence and cadence thereof, of a PUM or other stakeholders.

In some embodiments a pattern or set thereof, can have an agent bound to that pattern, which can control the one or more sensors, devices or systems generating the data sets that represent that pattern or provide one or more other systems with the operative state of that pattern.

These behaviors may then be evaluated for the one or more risks that they represent to a PUM or other stakeholder, for example, those correlated to time, locations, movements, other stakeholders and the like. In some embodiments such risks may be communicated to a PUM or other stakeholder, including a carer or may be stored in one or more repository and used, for example, to train one or more AI/ML systems, including agents.

In some embodiments such an AI/ML including LLM/LCM or agent based approach can result in the creation and deployment of an individual behavior model that represents a set of behaviors of a PUM. This approach facilitates a monitoring system employing such a behavior model to identify variations in that person's behaviors that can indicate one or more wellness, health or safety risks or concerns, which can then be communicated to that person, other stakeholders or other sensors, devices or systems. This personalization of monitoring can facilitate early detection of variations in behaviors that are indicative of changes in the wellness, health or safety state of the PUM. This can include the mental and physical attributes of the person. This personalized behavior model and the LLM/LCM based AI/ML system supporting it can adapt to the changes in the PUM state, for example over a period of time, where gradual deterioration or improvement can occur, such as for example when their eyesight may deteriorate or they recover from a procedure, for example knee surgery.

The early detection of any variations in behaviors can inform one or more sensors, devices or systems involved in the monitoring of the PUM as to possible responses that may mitigate or avoid any detrimental impact to them PUM from those behavior changes. This can include the reinforcement, through for example communications or rewards of behavior changes that have a positive effect on the wellbeing, health or safety of the PUM.

In some embodiments one or more systems may operate to provide context to the LLM, for example a PPE may be employed to ensure that the LLM does not generate behaviors that are simply impossible for a PUM to physically undertake. In some embodiments a suitable configured agent may also act as a to provide context using for example, an MCP, where for example the agent acts to provide constraints on the possible combinations an LLM/LCM may output, such as patterns or behaviors that are incompatible with the current condition of the PUM.

A further aspect of the personalized behavior representation is the identification of the cadence and sequences of behaviors and their potential and actual impact, including for example risk assessment, on the PUM. For example, this can include the combination of such representations in an aggregate form, where for example such representations are anonymized so as to evaluate optimal behaviors and sequences thereof. Using the same approach, the identification of behaviors and sequences thereof that can have a detrimental impact can be identified for multiple PUM with similar conditions or context.

In some embodiments, this can include mapping of such sequences to provide one or more representations of a wellness, health or safety journey for a PUM, which in turn can inform the decisions, responses or reactions to that journey by those stakeholders involved with the PUM. Such mapping, including the one or more data sets, patterns or behaviors of one or more PUM and can be stored in one or more repository and can provide a comprehensive data set for the training of one or more AI/ML system, including LLMs. For example, this can include the responses, impacts or effects of the one or more events or actions that have occurred and been measured by the SEE on one or more PUM to provide the capability of predicting likely outcomes for those events or actions, which may then be communicated to one or more PUM, other stakeholders or care monitoring systems, such as care hubs and care processing systems.

In some embodiments a behavior language can be human readable or machine interpretable data. In the example of a personalized behavior representation, this can be in the form of a set of encrypted tokens or other encrypted representation, such that only those authorized and authenticated stakeholders or their proxies are able to access the representation in part or in whole. In some embodiments, a behavior representation that includes a prediction of a set of behaviors over time, for example where a PUM health or wellbeing deteriorates to a substantial degree, such prediction can have limited distribution, for example controlled by other stakeholders, such as family of a PUM. For example, if a PUM has significant deterioration of their mental faculties predicted, the this may not necessarily be communicated to them, unless instructed to do so.

In some embodiments, the behavior language can represent the habits and regular activities of a PUM or other stakeholders, including their interactions. This representation can be used in one or more digital twin to identify trends and sequences of behaviors that are indicative of variations in the mental or physical state of the PUM or other stakeholders. These variations can be measured, and in some circumstances, may be communicated to the PUM or other stakeholders.

In some embodiments, one or more processes may be invoked, for example care processing or care hub, including one or more AI/ML systems, such as an LLM or agents, to determine, at least in part the start of a “conversation/sentence” comprising a set of behaviors, to identify a sequence of such behaviors to predict likely end states of these behaviors so as to assess and identify any risks or safety concerns, which can be represented by one or more metrics, for example risk metrics.

In some embodiments tokens representing patterns or behaviors, in whole or in part, may be evaluated, by for example, one or more AI/ML system, including LLMs or agents to identify the relationship between these patterns or behaviors, including movements therein, which are represented by tokens that can be evaluated by such an LLM as “words”, including those identified or classified as nouns, verbs and the like.

In some embodiments, the cadence of these behaviors, can be determined for example from measured or recognized patterns derived, at least in part, from the data sets of the one or more sensors, devices or systems. This can include the use of special tokens representing for example the start or end points of a pattern or behavior. This delineation of start or end points can involve one or more AI/ML systems, including an LLM or agent, where for example the tokens and vectors are aligned to identify which pattern(s) or behaviors(s) are currently being undertaken. This can provide further pattern analysis, in that there can be transition patterns, representing the transition from one behavior to another. In some situations, such transitions can form part of a sequence of patterns, where the behaviors form a recognized set of behaviors of a stakeholder. In some embodiments such sequences may form part of a repository that can be employed as a context for one or more LLM to ensure that the output of the LLM is consistent with the context, capabilities or intentions of a stakeholder.

The use of behavior sequences, including their relationships to time, including time or day, duration and other temporal data, for example that associated with a specific stakeholder, location, context and other characteristics, can inform one or more care hubs or care processing or monitoring systems as to the state of the stakeholder and any risks or concerns that may arise from such a sequence. These sequences can, in some embodiments, be mapped to one or more models of behaviors, such that the sequence can be evaluated as in sequence or out of sequence, matching or not one or more temporal windows for sequences, representing behaviors that are typical or not for one or more stakeholder and the like.

In some embodiments, one or more LLM can be employed to generate, at least in part one or more behavior languages, for example one that is representative of a specific stakeholder, location or context. These behavior languages can include syntax, semantics, contextual data and can be human and/or machine interpretable. This can include development or deployment of one or more models that for example include, relationships of tokens or sets thereof, context of token deployment or use of such tokens.

FIG. 5 illustrates use of a behavior language with an LLM, according to an example embodiment of the present disclosure. One or more data sets or patterns (501) are processed into tokens (502), suitable for one or more LLM (503). Such LLM may interact with one or more behavior models (504), including those instantiated by this or other LLM. The LLM (503) can interact with one or more behavior language expressions (506), which can be stored in one or more behavior language repository (505), where such repository can interact with behavior frameworks (507) or behavior knowledge base (508). In some embodiments, behavior language repository (505), behavior frameworks (507), behavior language expressions (506) and behavior knowledge base (508) can form, in part or in whole, behavior representation processing (510), which mya be includes in one or more monitoring systems or care processing systems. Each of the behavior representation processing entities, singly and in any combination can interact with a context or MCP (Model Context Protocol) server (509) that is configured to operate with the LLM (503). The Context or MCP server can be configured to operate with one or more third party or external systems (510), for example stakeholders such as insurance companies, service providers (for example, carer scheduling, delivery services, pharmacies and the like) weather or other third party services.

### AI/ML Including LLM State-Based Data and Representations

The combination of pattern language or behavior language tokens, supported by data sets generated by the one or more sensors, devices or systems present in a SEE can represent the state of that SEE or any stakeholders therein.

These states can be represented by tokens, where for example a behavior, for example sleeping has a state token representing that state, where the state of that token is, for example, quiescent. Such state tokens may have a relationship with a behavior token, for example through concurrent timing, though in some examples the state can encompass multiple behaviors. In some embodiments this can include generation of one or more state tokens.

These tokens can represent state and form sequences, for example from quiescent to activity or event or vice versa. In some embodiments, these states can have labels, which can be those of a behavior language, such as measured by a SEE. For example, a behavior sequence may be represented as sitting, standing, walking, for example to a specific location within a SEE, for example a kitchen, beverage preparation, carrying beverage, walking, sitting and beverage consumption. Within each of these labeled behaviors, the state may range from quiescent to active or event, and in some embodiments, this can include scalars such as numeric or descriptive. For example, if whilst walking and carrying the beverage, the state may vary from quiescent to a numeric value, for example four, on a scale of zero to ten, where ten is an event that impacts the wellbeing of the PUM and zero is the quiescent state. In this example such a variation may indicate a small spillage of the beverage.

In this example an AI/ML system, such as an LLM, may employ a model which is based, at least in part, on the observed behaviors of the PUM as they undertake their activities. Such a model may then be used to predict the behaviors, for example, if a person undertakes a sequence on a regular basis, for example at 11 am each day, then the model may include one or more representations of this behavior sequence.

In this example, the model, for example embodied in one or more digital twins, may be compared with the state exhibited by the PUM in real time as measured by the SEE, to determine, for example, such metrics as cross entropy loss and perplexity, which can represent the delta between the model predictions and the actualization of the PUM behavior at that time. These metrics may inform, at least in part, the one or more monitoring systems employed to undertake analysis of the PUM's behaviors, such as care hubs or care processing systems. Such variations can provide indicators of the variance of the PUM behavior, for example where there is a deterioration in the health or wellbeing of the PUM.

One aspect of this approach is the use of sparse data sets indicating state changes, to indicate at the earliest possible time that such a state change represents a potential or actual action or event that can have a detrimental or other impact on the PUM.

In some embodiments, such indications may be used to communicate with the one or more sensors, devices or systems within a SEE to vary their configurations or request further data sets, including increased granularity, timeliness or other parameters, such that this additional data can provide additional verification, validation or further detail of the activities of the PUM, to at least in part, evaluate the state change.

For example, in some embodiments such techniques as Softmax can be employed for the evaluation of the one or more tokens representing the SEE data sets, including those token representing patterns or behaviors including the languages thereof.

Models can be created using data generated by one or more sensors, devices or systems present in a SEE, where such data that exceeds one or more thresholds and where such thresholds are, in whole or in part, representative of the state of the measured environment and any humans therein such that these models can represent events, actions, occurrences and the like which are stateful. These models can be used, for example, to evaluate SEE data sets, including patterns or behaviors, to identify actions, events or occurrences involving the PUM or other stakeholders including interactions between them.

In some embodiments, changes in the state variations, for example from zero to six on a scalar of zero to ten, where ten is high, may cause one or more monitoring system to communicate with the one or more sensors, devices or systems in a SEE with a notification, for example a configuration variation, for those sensors, devices or systems to enhance their data quality, through for example, increasing granularity, frequency, detail or using further sensors, devices or systems capabilities, for example configuring a camera for a visual image rather than an edge identification to, for example increase the predicative capability of a model or provide monitoring systems with more detailed data so that the state of the PUM or other stakeholders may be more accurately determined.

Tokenization and embedding are techniques used to represent natural language, including its meaning, in a way that can be processed by computers. They are fundamental concepts in Natural Language Processing (NLP) and Large Language Models (LLMs).

In some example embodiments of LLMs that are intended to operate on human interpretable language, tokenization is employed to break down text into individual units, called tokens. Tokens can be words, characters, or even sub-words (smaller units within words), for example syllables, phonetics or other character groupings. The goal of tokenization is to convert text into a format that can be easily analyzed and understood by machines. There are several types of tokenization, including, for example, Word-level tokenization, where each word in a text is treated as a separate token, Sub-word-level tokenization, where words are broken down into smaller sub-words (for example, “unbreakable” becomes [“un”, “break”, “able”]), and character-level tokenization, where the text is converted to individual characters or other quantization's of the text, such as syllables, phonetics or the like.

Similarly, when dealing with data sets generated by the one or more sensors, devices or systems present in a SEE, including patterns or behaviors, these may be processed using one or more tokenization techniques to create tokens that can be understood and managed by machines, for example those computers operating an LLM.

This tokenization process can include differing segmentations including quantization's or levels at which such tokenization occurs, for example, if a behavior comprises a set of patterns in a specific order, and such patterns comprise data sets in a specific arrangement, then the tokenization processes can operate at any or all of these levels.

In some embodiments one or more sets of quality metrics can be applied to the one or more data sets generated by the one or more sensors, devices or systems, which can include relationships between these data sets, the patterns formed from them and the behaviors such data sets or patterns represent with one or more predictive models, for example those generated by one or more LLMs, to determine or evaluate accuracy or other characteristics of these data sets. This can, in some embodiments, include the personalization of such models to one or more stakeholder, for example a PUM, where the monitoring systems, including care hubs or care processing systems can invoke or deploy such a model. This can include quality metrics and can involve the further refining of those metrics or the accuracy of the model, which can be used in the evaluation of data sets generated by the SEE.

In some embodiments, one or more monitoring processes, including for example care hubs or care processing systems, including those incorporating or using one or more LLM, may request further data sets from one or more sensors, devices or sensors present in a SEE, including from those that have not provided data sets to these monitoring processes at that time, to at least in part, determine the certainty of such data sets, including validation, verification, authentication or other characteristics of these data sets, for example represented by one or more metrics.

In some embodiments one or more AI/ML systems, including LLMs can be employed to identify, determine or predict, at least in part, unknown or unrecognized patterns, for example using a pattern language framework. For example if a set of data is identified as a potential pattern or in a sequence of patterns, there is a gap, then an LLM may predict that such data is a pattern and compare this with, for example known patterns or may postulate that there is a missing pattern in a sequence of patterns and then, for example request that the one or more sensors, devices or systems provide data sets that can correspond to that pattern, for example using temporal delineations.

This can include, for example quantization or tokenization of one or more states of these data sets, the patterns or behaviors in which they may be included and the state of the SEE or the stakeholders therein. For example, there may be one or more metrics employed for the representation of the accuracy of the individual or collective states of the SEE and the data sets, and arrangements thereof, including patterns or behaviors, in whole or in part.

In some embodiments, metrics for evaluation by an LLM of non-textual data sets, including for example those generated by one or more sensors, device or system and represented, in part or in whole by patterns or behaviors, can be based on the relationship between segmentation of those data sets and their evaluation. For example, if the data sets are segmented into small or narrow units or tokens, then although the precision when can be high, quality is likely to be low. The relationship between the segmentation of data sets and the metrics of their evaluation can be crucial to the subsequent use by, for example, an LLM and the accuracy of any outputs from that evaluation. This segmentation or tokenization can include, for example, measuring periods between data sets or elements thereof, that includes same or similar data, where such measurements can be represented by tokens, and in some embodiments, specialist tokens, such as quiescent tokens, potentially representing data sets or periods where any change is minimal or below one or more thresholds.

The precision of the metrics can be correlated to the quantization, segmentation or tokenization of the underlying one or more data sets, for example, if one or more sensors, devices or systems tokenize, for example as patterns, those data sets that match the criteria for such patterns, and those patterns can form a sequence, which can form part of a pattern language, to which typical LLM techniques may be applied.

This can include, in some embodiments the use of vectors to represent these one or more states or the metrics thereof.

### Embedding

An embedding is a mapping of a discrete, categorical variable to a vector of continuous numbers. In the context of artificial neural networks, embeddings are high-dimensional, learned continuous vector representations of discrete variables. One especially useful characteristic of embeddings is that they can place entities that are similar, closer to one another in the embedded space.

Neural network embeddings are useful because they can reduce the dimensionality of categorical variables and meaningfully represent categories in the transformed space.

Embeddings can be used in current LLM systems, where embeddings are numerical representations of words, phrases, or other text units in a high-dimensional space. The goal of embeddings is to capture the semantic meaning and relationships between different text units of a human interpretable language. Representing meaning as numeric values in a consistent high-dimensional space allows for manipulation of this data using techniques such as linear algebra operations, making it possible to consistently adjust the meaning of a text unit, in its embedded form, based on other surrounding text units, or its position within a larger text or other contextual parameters. This representation of meaning using embeddings, or semantic encoding, is usually achieved through neural network supervised learning.

In many current LLM embodiments, language data input, internal processing and output are all used in their embedding form, which is more suitable for mathematical processing, while consistently maintaining their categorization and meaning representation. The process usually starts by breaking text into tokens, which can be words or sub-words, following different possible strategies. Then each token gets mapped to a learned vector, usually, via an embedding matrix. Transformer layers then modify these embeddings based on context. These modified embeddings are typically used to, for example, predict the possible next word, in after a word sequence presented as input, which is the common basis for generative LLMs. Tokenization and embeddings usually start by preprocessing the text data using tokenization techniques to convert it into a suitable format for analysis. Then embeddings are generated by mapping each token to a high-dimensional vector space that captures its semantic meaning and relationships. This is usually done by using a pre-existing vocabulary of all the supported unique tokens and their mapped high-dimensional vectors. The embedded text data is used to train models or as input to pre-trained models, in order to obtain, for example, inference results.

Visual, audio or multi-modal LLMs follow a similar process, where discrete tokens are obtained by extracting small parts of the input content, using one or more of multiple techniques (Patch-Based Tokenization, Codebook/VQVAE, Convolutional Feature Maps, Neural Audio Codecs, Spectrogram, Contrastive Learning, and the like). These techniques are used for training the embedding AI models, which can then be used as part of the LLMs data flow.

The embedding concept, which allows categorization and numerical processing of concepts and their relationships, generalizes naturally to other domains, such as, for example, molecular biology, recommendation or monitoring systems, such as those described herein. In some embodiments it is possible to apply tokenization and embedding to learn or represent classification and relationships including those based on data sets from one or more sensors, devices or systems including sequences and other arrangements of measurements generated by multiple sensors, devices or systems within a SEE. In these cases, instead of processing each sensor data stream independently, embeddings can map different sensor, device or system modalities into a shared space, for example a semantic space, where similar SEE or PUM states cluster together, regardless of which sensor, device or system detected them. For example, a fall detected by an acceleration spike (accelerometer), sudden altitude change (barometer), and impact audio (microphone) can all map to nearby points in embedding space, enabling efficient and robust multi-sensor confirmation of the fall. In some embodiments this can be combined with AI/ML self-attention mechanisms, such as the one in the LLMs Transformer architecture, where the system can learn which sensor, device or system, including sets or arrangements thereof, are most important for each one or more state, event, occurrence, pattern or behavior type.

Tokenization processes can be applied to non-human readable data sets, including for example data sets generated by one or more sensors, devices or systems of a SEE or pattern languages or behavior languages based on such data that can include classifications that represent the one or more states, patterns or one or more behaviors of a person under monitoring. The vocabulary for such processes can include mappings of data sets generated by the one or more sensors, devices or systems present in a SEE, including arrangements of such data sets, for example as patterns or behaviors. This includes motion-based data sets such as those generated by, for example, a set of motion detection sensors, a Personal Physics Engine (PPE) or a combination thereof.

A further aspect of this approach is the timing of such data sets, in that each of the sensors, devices or systems employed or present in a SEE can share a common time base, such that each of these datasets, patterns or behaviors occurs at or over a specific time-period, creating a context for these elements. This contextual data can form part of the tokenization, embedding or high dimensional vector space. The addition of time to the embedding, in one or multiple embedding steps of the data process and analysis, facilitates the capture and classification of temporal dynamics, such as, for example, a sudden fall compared to a sudden movement of a PUM.

Sensor, device or system data sets, including those that are represented as signals can be combined using multiple possible techniques before using embedding on the combined signal. They can also be embedded separately, and these individual embeddings can be fused together, through for example, concatenation, generating a single embedding, in a single shared space and the like.

In some embodiments, data sets, including streams from one or more sensors, devices or systems may be segmented for further processing. This can include the use of one or more techniques to create individual data segments, each of which can have separate identity or be identified by the data itself. For example, these segments can be tokenized or form embeddings that are specific to the state of the PUM. Such segmentation can include quantization's based on, for example time, data content, thresholds, triggers, events, sequences, patterns or behaviors.

In some embodiments, one or more sensors, devices or systems may include one or more AI/ML systems, including agents, that can identify, segment, classify, categorize or organize the data sets generated by them, for use by, for example, an LLM or other AI/ML systems. These data set processing techniques may be based on, e.g., time, data set contents, data set state, the choice of algorithms used for the original data generation, context, or other sensor, device or system.

For example, a sensor, device or system may generate a continuous set of data, which can be segmented according to a time specifications, for example each X seconds an EOS (End of Segment) data pointer is inserted into the stream, which in some embodiments may be represented by a special token. Alternatively, the configuration of the sensor, device or system may include specifications that govern the operations such that data is only transmitted on a timed basis, except where the data exceeds one or more configured thresholds.

In some embodiments, there can be a vocabulary of tokens, for example in a token repository, that represent the one or more data sets, patterns or behaviors as well as special tokens that represent, for example state or events. These tokens can be personalized for a specific PUM in a specific location, for example a SEE. For example, this personalization can include using tokens representing a behavior or behavior pattern of a PUM, such as coughing, shaking of a limb, or unsteady footfall whilst walking.

Some sensors, devices or systems can be configured such that if the data generated or received by such sensors, devices, or system, exceeds or varies from a specified set of values, then the data may be, for example communicated to one or more other system including care hub, care processing or one or more AI/ML system. In this example, such data may be “raw”, for example, without any segmentation.

FIG. 6 illustrates an example SEE which identifies state of data sets, according to an example embodiment of the present disclosure. One or more data sets (601) can be generated by one or more sensors, devices or systems present in a SEE. The SEE may include one or more watchdog (602) configured to identify the state of these data sets. For example, the watchdog may ascertain whether such sets are quiescent or not, using a state manager (603). These data sets (601) together with any meta data, such as determined by the watchdog, can be communicated to an alignment and evaluation process (604) which interacts with an LLM (605). The LLM (605) interacts with a pattern framework repository (607). The pattern framework repository may together in combination with sensor pattern frameworks (606) further communicate with an alignment and evaluation process (608). A further LLM (609), which can interact with a pattern repository (611) to identify or generate patterns (610). In some embodiments these patterns may be communicated to one or more mapping processes (612) to generate one or more tokens (613).

The use of thresholds or other configuration specifications that, in whole or in part, determine whether the generated data is communicated to another sensor, device or system, can be used to reduce data communications and any resources used by such communications, including for example battery, CPU, memory, storage and the like, whereby for example, only when the state of the monitored environment varies does the sensor, device or system undertake communications. In this example, if the state of the environment is quiescent, then the one or more sensors, devices or systems only communicate when the generated data stream has one or more data sets that represent a change in that state.

In all these examples, the segmentation of a data stream or set into discrete segments can facilitate one or more AI/ML system, including LLMs, to use these data sets as “words” or “tokens” for example, to generate one or more representations of the state of the environment. Such segmentation or tokenization can use one or more techniques, such as byte pair encoding, Huffman encoding, 3D-cell based encoding and the like. There can be a founding relationship between the segmentation of these data sets and the effective and accurate operations of one or more LLM, including agents, in that if the segments are of a type that matches or aligns with the LLM embedding processing or training, such that the determination of the vector relationships in the higher dimensional space is well supported by this segmentation, and consequent tokenization, then the accuracy, reliability or utility of the LLM for that data set can be optimized.

These quantization's, segmentations or tokenization's may include data sets from multiple sensors, devices or system sets with predetermined relationships or dynamic relationships, for example where one sensor has configured or configures another sensor such that the data set from the one or more sensors is used by one or more embedding techniques, including tokenization. This can include, in some embodiments, the use of multiple inputs from multiple sensors, devices or systems in a time window, for example one with a start time or of a duration. This can include, for example, the state of the data sets generated by the one or more sensors, devices or systems, for example when such data set exceeds a threshold and represents, at least in part, an alert or event or, in whole or in part a pattern.

In some embodiments, sensor, device or system data sets may form a stream where the sensors, devices or systems continuously generate data based on their configurations. This data can have one or more meta data, including for example time data, such that stream can be segmented into quantized data sets using such meta data or using other appropriate segmentation or tokenization, for example where the sensors, devices or systems meta data includes segmentation data. Some sensor, device or system configurations may include such quantization specifications, such that the data is segmented by, for example time, quantity, one or more attributes of the data or specifications received from one or more other sensor, device or system.

LLM may output specifications for deployment and configuration of one or more agents to modulate the incoming data streams for that or other AI/ML including LLM. The agent data sets can, in some embodiments, be substituted for or modify the data streams from the one or more sensors, devices or systems of the SEE, such that the volume of tokens for the embedding is significantly reduced, for example when the state of the SEE or PUM is quiescent.

In some embodiments, there can be special token sets which can represent one or more data sets, patterns or behaviors that are observed during the monitoring of an environment. For example, these can include, start, end, state, including one or more specific states, such as fall, trip, breathing anomaly or other care, wellness, health or safety states, reset tokens and the like.

In some embodiments, one or more AI/ML systems may be employed by the one or more sensors, devices or systems to undertake self-supervised learning, which can result in the data sets generated by such sensors, devices or systems being segmented and tokenized in a manner suitable for evaluation or use by other AI/ML systems, including for example LLMs or agents.

For example, in some embodiments, combinations of, for example, sensor, device or system data streams, one or more PPE data sets, including streams or one or more pattern data sets, may form sets of tokens that can be interpreted by one or more AI/ML system, including LLMs. For example, if the LLM employs a context/MCP server, such tokenized data sets may be evaluated by the context/MCP server in combination with the predictions or other outputs of the LLM to establish the validity of the LLM outputs or identify relationships between these tokens and the LLM outputs.

One aspect of this approach is establishing the optimum granularity for data sets, patterns or behaviors, such that one or more monitoring systems, including care hubs or care processing, can identify any variations in the state of the behaviors of a PUM, for example moving or transitioning from a quiescent state to another non quiescent state or form one behavior to another. For example, one or more AI/ML systems, including for example LLMs may predict the likely transitions, however if the granularity of the data sets, patterns or behaviors represented by tokens is inappropriate, in that is too detailed or to abstract, the advantages of the LLM may not be beneficial or timely. This can include data sets, patterns or behaviors generated, in whole or in part, by the one or more sensors, devices or systems present in a SEE, including for example a PPE.

This can include determination of any bias within a data set, pattern or behavior, where certain, for example, sensors, devices or systems generated data sets, patterns or behaviors are favored in comparison to those of others. For example, the one or more weightings employed may be purposefully biased to one or more sensors, devices or systems, where for example these have an established record of accuracy, timeliness, validation, confirmation or other characteristics, the confidence of these measurements may be high and consequently represented as such by the weightings. Similarly, if for example, one or more sensors, devices or systems have generated data sets, patterns or behaviors that, when evaluated, have a poor record of accuracy, timeliness, verification or other characteristics that reduce confidence in those measurements, the weightings may represent this.

In some embodiments, data sets may from part of open sets, where for example the addition of one or more further sensors, devices or systems and the generated data sets thereof may be included in such an open set. For example, if the open set is part of a verification process, where for example data from one or more sensor belonging to that set has, at least in part, initiated a verification process, for example by a care hub or care processing system, then further sensors and their data may form part of this set to facilitate verification of the initial trigger event. These opens sets may, in some embodiments, form part of the configuration of the one or more sensors, such that, for example a sensor may include relationships with multiple open sets, for example those comprising other sensors that are collocated or capable of generating data that can, for example, be used to verify the output of one or more other sensors.

In some embodiments, one or more sensors and the date generated thereby, can form part of a closed set. For example, a closed set can be a set of sensors and data that represents a pattern. There can be one or more process that selects which sensors, devices or systems and the data sets thereof form such a closed set. For example, if a pattern has been detected and repeated sufficient times to confirm that it is reliably repeated, then one or more processes, including for example care hub or care processing or other monitoring may configure a closed set of those sensors, devices or systems that contribute data sets to that pattern and the data sets thereof. This can include the recognition of such data sets by one or more AI/ML systems using a range of techniques, including use of BERT, skip gram, masking, data shuffling, hidden Markov models or the like.

In some embodiments one or more monitoring system including for example, care hub or care processing, may identify an initial token, described as an edge token, which can indicate that a sequence of tokens will follow, for example a set of tokens representing a pattern or behavior, or indicating a change in state, for example from quiescent to non-quiescent. These initial tokens can indicate, for example, edge state changes or transitions and can, in some embodiments form the input to one or more AI/ML system, including LLMs, that can then predict, based on the tokens the most likely set of tokens that will follow, which in some embodiments, may be passed to one or more digital twins.

In some embodiments, one or more AI/ML system may be employed, at least in part, to determine one or more weightings that can be applied to the one or more data sets, patterns or behaviors generated by the one or more sensors, devices or systems of a SEE. This can include, for example the use of risk and other metrics such that the weightings include current, potential or predicted risk or other metrics. These weightings and the risk and other metrics thereof may be retained in one or more repository, for example a graph database and may for example include sets of relationships with such weightings and their application by, for example, one or more AI/ML system including LLMs.

In some embodiments, multiple encoders can be employed to, for example, process one or more data sets, which for example may form, in whole or in part, one or more pattern or behavior. For example, a set of encoders may operate on the differing data representations, for example one set may operate at the data set level, a second set on the pattern level, where the data sets may form, in whole or in part, the patterns or on the behavior level, where such behaviors may comprise the data sets or patterns in any arrangement.

In some embodiments an LLM may be used to develop or operate on a model that is personalized to a specific one or more stakeholders, for example a PUM, or those stakeholders involved in their care, well being or safety. Such a model may then undergo anonymization and be aggregated with similar models of other stakeholders who have common characteristics, such as age, health condition, location or other attributes in any arrangement, to form a meta-model than can be employed, for example, for the identification or determination of further meta patterns or meta behaviors.

In some embodiments, one or more pre-trained models may be deployed, for example, for a specific stakeholder, including a PUM or a set thereof. In some embodiments, for example, a set of stakeholders may have shared characteristics, including for example their location, for example a care facility or campus. Such a model can then be fine-tuned, using for example, specific datasets to adapt to new tasks, characteristics, locations or domains, including for example, individual SEEs or PUMs or other stakeholders.

Such an approach can, in some embodiments, include models that form the basis for one or more health anomaly detection systems, where detection of, for example, changes in movement patterns or other characteristics of the data sets, patterns or behaviors, may indicate one or more health issues, such as Parkinson's disease or stroke.

This can include for example identification or recognition of emotions, based at least in part, on body movements, including parts thereof, for the detection, recognition and potential support or remediation of one or more emotive states of a stakeholder, for example, happiness, sadness, fear/distress, or anger and the like. This can be of particular significance in identification of incentive misalignment where for example one stakeholder, for example the PUM exhibits stress, sadness, fear or other negative emotions in the presence of another stakeholder, for example a carer.

The employment of personalized or specialized models can support further interactions and communications with or about a PUM, including for example, rehabilitation or therapy, where for example personalized rehabilitation programs can be based, at least in part, on individualized movement patterns, behaviors, intentions or goals. This can include sets of metrics that, for example employ one or more sensors, devices or systems present in a SEE to identify or measure the activities and progress for such programs. This can include the development or deployment of one or more specialized LLMs, including models that can provide a personalized or specialized capability for a particular condition, for example, including therapeutic or remedial programmatic elements. This can include the use of one or more context or MCP servers or fine tuning to optimize the performance of the LLM in a specific context, for example recovery from hip or knee replacement.

For example, an LLM can be applied to intention detection, where a stakeholders intention indicated at least in part by one or more specific movement(s), for example, “greeting”, “getting up”, “sitting down”, “stumbling” or the like, is predicted by the LLM, using for example, digital twins, game theory systems or other AI/ML systems.

These intentions and the data sets, patterns or behaviors that represent them may from part of a model, for example one generated, at least in part, by an LLM.

FIG. 8 illustrates training of models in an example SEE, according to an example embodiment of the present disclosure. A SEE, that can include a PUM or other stakeholders (801) generates data sets, including for example those form a PPE (802), which can be communicated to one or more LLM (803), which may invoke, deploy or use one or more digital twin (804) or one or more game theory engines and the games thereof (805), to train or generate one or more models, for example, including pattern models (806), behavior models (807), state models (808), action models (809), intention models (810) or response models (811) in any arrangement. These models can interaction with one or more agents (812) in any arrangement.

FIG. 9 illustrates an example system, according to an example embodiment of the present disclosure. The example system can include one or more input systems (901) and one or more output systems (902), which communicate or interact with sensor data models (903), pattern models (904), behavior models (905), response models (909), action models (910), intention models (911), temporal models (907), sequence models (908), AI/ML systems (912), one or more LLM (913), one or more RAG (914), one or more context or MCP servers (931), one or more game theory engine and games thereof (917), one or more knowledge base (915), one or more repositories (916), pattern frameworks (922), patterns (923), one or more pattern language (924), behavior frameworks (925), behaviors (926), one or more behavior language (927), one or more SEE (928), one or more data sets (929), one or more PPE (930), state models (906), one or more states management (918), one or more agents management (919), one or more agent instances (920), one or more agent frameworks (921) or one or more predictive states (917) in any arrangement.

### Agent Based Approach

In some embodiments, one or more AI/ML, including LLM, systems may operate with one or more agents which can be deployed in or by the one or more sensors, devices or systems that are present in a SEE. An AI/ML Agent includes sufficient autonomy to undertake the one or more tasks for which it is configured. These AI/ML agents can operate, to undertake one or more operational tasks or processes. Including for example reasoning, decisions or planning, in accordance with their configuration and can include retaining and persisting such operations or processes, including developing or executing a specified set of operations in support of monitoring a SEE and any stakeholders therein, including a PUM.

In some embodiments agents may be configured, including dynamically, for example a response agent configuration may use a model of a SEE and a PUM, representing the world in which the agent is operating, to make decisions. For example, operations of one or more the sensors, devices or systems, where such an agent may operate as part of such, can for example, improve, validate, verify or otherwise undertake monitoring operations of those hosts or the agent itself.

In some embodiments an agent can be generated, for example using an agent framework, based on one or more data sets generated by the one or more sensors, devices or systems present in a SEE, which can include patterns or behaviors that have been previously recognized by, for example one or more monito ring system, including one that generates such agent. The generation of such agent can include the configuration of the agent, which can include input management, processing, including decision making, and outputs.

The selection criteria for the generation of an agent can include the recognition of the occurrence of a previously recognized pattern in the data sets generated by the one or more sensors, devices or systems of a SEE, for example where such pattern is stored in a repository managed by the system generating the agent, for example a monitoring system. This recognition can include patterns generated by, for example, a digital twin which, using the data sets generated by the one or more sensors, devices or systems of the SEE, has predicted, for example using AI/ML or agents for that purpose, an activity, event or occurrence which match the selection criteria. For example, if the PUM being monitored starts having a coughing experience measured by the one or more sensors, devices or systems of the SEE, and such measurements represent a known pattern for that PUM, then an agent can be generated, configured and deployed to, for example, adjust the HVAC systems to filter the air flow, advise the PUM to sit or remain sitting, dispense cough syrup or cough medications through a dispenser or inform through one or more devices in proximity to the PUM other possible remedies, such as holding their head in a particular direction. The agent can also output communications to, for example, to the device of a carer, alerting them of the current situation.

The recognition of a specific state of the PUM, represented by one or more patterns or behaviors and the dynamic generation and deployment of one or more agent configured to assist, mitigate or resolve that state to the benefit of the PUM significantly improves the efficiency of operations of an AI/ML system involved in the monitoring of the PUM. This can include the use of preformatted token sets being provided to the agent, resulting in significantly less resources being consumed in the resolution of the state of the PUM.

For example, an agent can be employed to evaluate the same set of tokens representing a specific sate, for example a PUM having a coughing episode, however if the PUM has an episode on day one, and then again on day three, the agent can be configured to evaluate the differences as the context for the coughing is different, at least in time, and potentially in other manners, such as location, the PUM physical movements and the like. In this example, such agent may generate outputs that are different for each of these episodes based on the same set of tokens with different context data.

In some embodiments one or more agent can be generated, configured and deployed based on one or more data sets generated by the one or more sensors, devices or systems of the SEE. For example, a LIDAR operating in the SEE can output data in a LAS or LAZ format, which the agent is configured to process and based on this processing can operate to generate an output, for example the configuration of another one or more sensor, for example an image capture sensor, or the deployment of another agent. The data from the image capture sensor can be evaluated, for example by the same or differing agent, to validate the data set of the LIDAR, for example to confirm a change in orientation of a PUM, for example rising from sitting to standing, dropping the head into their hands, or leaning back in a sitting position. These changes may represent a change in state of the PUM, including a health, wellness or safety event which can be communicated to one or more monitoring systems or other stakeholders, including carers.

In some embodiments an agent can be generated to monitor a sensor generating data in a SEE. This data can be in the format generated by the sensor and the agent is configured to operate on that data. The agent can be configured with a set of variables, for example thresholds, vectors, triggers or one or more algorithms that measure the incoming data to generate a model of that data. For example, a sensor data stream that comprises audio can be evaluated to identify variations based on audio frequency, volume, rates of change or specific data, such as human verbal language.

An agent host is the environment in which an agent operates, providing that agent the resources needed for the agent's operations. This can include computing resources such as memory, processing, interfaces and the like and can include one or more sensing capability, tools to perform actions, to match the configuration of the agent.

In some embodiment, configuration of an agent can include specification of one more characteristic of the agent and agents operations, including specifications of objectives or goals, data set acquisition or processing, or model parameters, including weights. In some embodiments, these model parameters can include AI/ML model parameters that result from a fine-tuning process. For example, this can include PUM state, SEE data or groups thereof, including patterns or behaviors, which can be represented by tokens suitable for processing by one or more agents.

In some embodiments, configurations can include goal or objective based agents, where an objective is stated as part of the configuration of the agent and the agent, including the agent host, has sufficient data, for example in the form of data sets, patterns, behaviors and the like, to generate decisions for the operations of those agents, hosts or provide input to one or more other systems, including one or more sensors, devices or systems such as for example care processing or care hubs, which can include one or more AI/ML systems including LLMs. In some embodiments, such goal setting may involve, in whole or in part, one or more game theory modules for deployment of one or more games as part of the configuration, deployment or operations of that agent. This can include the determination of an optimum approach to undertaking a monitoring or other activity or task, such as communication or interventions, where for example one or more game theory games is employed as part of the evaluation of that optimization or acts to undertake one or more configurations of one or more sensors, devices or systems. For example, this can include changing the configuration of one or more sensors to increase the fidelity, frequency or efficiency of sensors monitoring the PUM, which can include variations in the focus or objectives of the monitoring, including those worn or carried by the PUM.

An optimal state can include, for example, the state of the health, wellness or safety of a PUM, where such state is represented by one or more specifications. These specifications can include one or more thresholds or metrics, for example risk metrics or other specifications of the PUM conditions or situations.

In some embodiments an optimal state can include the efficient and effective operations of the monitoring systems where the resources for that monitoring are available and deployed in a manner that optimizes their operations. For example, this can include the selection, configuration or deployment of the one or more sensors, devices or systems for the purpose of monitoring an environment such as a SEE. This can include the selection, configuration or deployment of one or more AI agents for the purpose of that monitoring.

Such optimization can include each agent or further agents undertaking learning, based at least in part on feedback, including that of the one or more stakeholders, for example expressed directly through one or more communications, such as voice, visual (including textual or written) or haptic actions, where the operations of the one or more agents and their hosts can use one or more knowledge bases developed, at least in part, from the operations of such agents, sensors, devices, or systems present in a SEE.

There can be a number of approaches to determining an optimal operation, action, configuration or other task or activity that can, for example, involve the monitoring of a SEE, which includes the configuration, operation or management of one or more sensors, devices or systems or interacting with stakeholders present therein, including a PUM. One approach is using utility theory, which can be part of decision theory, where the intention is to achieve an objective and to do so in an optimal manner. For example, this can include obtaining the highest possible score, based on one or more metrics, whilst ensuring all scoring exceeds an average of scores for that operation, where the probability of such scoring can form part of the determination of the utility.

In some embodiments an approach can include optimizing a risk metric, where for example there may be multiple risk metrics for different PUM conditions, their actions (or inactions), occurrences or events, including those measured or predicted. In this example the overall risk metric for a PUM is optimized when it is minimized.

One aspect of the deployment of one or more agents in a SEE is the negotiation between the agents for access to or control of data sets or processing resources, including decisions, between them. In some embodiments, if Agent A has a data set and is configured to monitor a specific aspect of a PUM, for example breathing, and Agent B is configured to operate in the same SEE and monitor the same PUM for another health, wellness or safety aspect, for example heart condition, these two agents may need to share their data sets and potentially collaborate to generate an outcome. For example, if the breathing data set and the heart monitoring data set both are identified by each of the agents as representing a potential health, wellness or safety issue, then they can combine their outputs for communication to, for example, a carer, a system (for example care processing, care hub) or another party, for example emergency services, based at least in part on the measured severity of the individual and collective conditions.

In the same manner each agent may generate an output, which is based on a monitored data set, for example one generated by a SEE, however another agent may have a data set that contradicts or does not support the first agent's data set. In this example, a further system, which can be or include other agents can be invoked to reconcile the differing data sets and generate an appropriate outcome.

In some embodiments, an agent (H) can be configured and deployed to operate to reconcile differing agent operations, where for example an Agent C is providing a data set and Agent (D) is also providing a data set, both data sets representing a current situation in a SEE involving a PUM. In this example, the evaluation of each data set and the priority of the data within those sets can be evaluated by agent (H) to generate a combined output or decision based on these data sets. This can resolve situations where, for example, a sensor is generating a data set with low certainty, and the agent (G) configured to represent that sensor data has a high priority and a second sensor generates a data set with high certainty and is represented by an agent (J) with lower priority. In this example, agent (H) may represent the data sets of both agent (G) and agent (J) in a format that includes meta data representing the priority and certainty of the data sets and agents. In this manner agent (H) can be configured to, as part of a monitoring system, generate an output to vary the configuration of the agents or sensors, or invoke further sensors to validate the data sets of agent (G) or (J).

One aspect of the deployment of the one or more sensors, devices or systems is their physical or logical location within an environment, for example a SEE. The calibration of the environment can involve establishing specific locations for such sensors, devices or systems, such that their sensing capabilities are optimized for observing or measuring the one or more stakeholders present or anticipated to be present, in that environment. This can include ensuring that there is sufficient coverage between sensing capabilities, such that there can be multiple data sets generated for any time period, event or activity, including with differing sensing capabilities, for example visual, audio, RADAR, LIDAR or haptic.

For example, an environment can be mapped using one or more sensors, devices or systems to identify the location within the environment of the fixed structures and furniture therein. This mapping can be represented in a digital twin or other repository and, for example using one or more AI/ML systems the various possible common PUM movements can be predicted or calculated, such as from bedroom to bathroom, sofa to kitchen, and the like. These routes can inform the placement or configuration of the one or more sensors, devices or systems within the environment. This can then be represented in one or more digital twin which can form part of the monitoring of that environment.

In some embodiments this can include worn or carried sensors, devices or systems where such portable units can provide data sets that can form, in whole or in part, one or more patterns or behaviors. Such patterns or behaviors can include the context of their location or time, such that for example, these can inform one or more AI/ML systems as to these contexts or provide state-based data to one or more other systems, including for example care processing or care hubs. Such contextual information may inform, at least in part, the determination of the type, characteristics, or configuration of an agent, including those based on agent frameworks that may be deployed on one or more sensors, devices or systems. For example, this configuration can include one or more risk metrics which may be used to inform that configuration. For example, a camera may be focused on the movements of a PUM limb, such as a hand or arm to monitor jitter or other involuntary movements.

In some embodiments, worn or carried devices or embedded sensors, devices or systems can include an agent, which can be instantiated as software or hardware in any arrangement. These agents can include one or more AI/ML capabilities, such that the agent can form part of a network of agents and can include one or more AI/ML systems.

Agent actions can be based on one or more action plan model, which can include for example, one or more pattern, behavior, state, action, intention, response, or other models, for example, representing the predicted or most likely actions or activities, including patterns or behaviors, of a PUM to identify one or more instances where AI/ML assistance can or could be provided. Such agent provisioning can be facilitated through, for example, sets of agents on one or more devices, which can include a SEE or specialist versions, for example those configured for a carer, relative, friend or other stakeholder.

### Agent Efficiency

In some embodiments, Agent (A) generates token set (a) and communicates token set (a) to Agent B, where agent A is configured to undertake operations that are a subset of operations of Agent B, and as such token set (a) is a subset of token set (b) of Agent B. In this example, the token set (a) may be used by Agent B, in whole or in part. This use of common sets of tokens, which can represent the state of a PUM, such as a pattern or behavior, can improve the efficiency of the operations of the deployed agents, including configuration and deployment of other agents, for example where agent A initiates, at least in part, deployment of Agent B, such that the processing of the tokens by the agent results in minimal resource use for that operation.

In some embodiments, as the patterns and behaviors are representations of the PUM state and as such by their nature are repeated, the difference between episodes of these occurrences can include context, severity and timing and as such the agent outcomes may differ based on these variations. However, the efficiency of the agent operations, particularly in terms of resource consumption can be reduced, which can facilitate these agents being hosted, for example, closer to the edge. For example directly in the sensors, devices or systems of the SEE employed to monitor the PUM. This is particularly significant for mobile, worn, carried or embedded sensors, devices or systems where such resource availability is limited.

In some embodiments, deployment of an agent is, at least in part, dependent on the ability of the agent host environment to support the agent operations. This includes resources for the agent operations and time for those operations. For example, resources can include computing resources, such as processing, memory and the like, interfaces to data sets, for example sensors, interfaces to communications, for example for agent outputs and the like. For every time period in which an agent operates there is a continuing resource requirement to accommodate these operations.

One approach to achieving agent operating efficiency is the reduction of tokens being processed by the agent, which in turn reduces the burden on the computing resources. Another approach is separation of the model from the agent, such that the agent can use the model for agent operations, however the model is not using the computing resources of the agent. Further agent operating efficacy can be achieved through segmentation of the data sets generated by the SEE, in which sets of agents operating, for example, as a hierarchy, where there is one agent that is configured to manage the operations of a set of agents. For example, in a SEE with multiple sensors capable of generating a range of data types (for example audio, video, haptic, temperature, humidity and the like), each sensor can have an agent configured to operate on the data sets of that sensor. This can include multiple agents operating on single data sets, for example a video sensor, where each agent or set of agents is configured to recognize different features of the data set or agents are configured to operate as a multiplex, for example on a time slice or data quantity segmentation basis.

In some embodiments, an agent may use one or more models, some of which are hosted in one or more digital twins, where each of these models is configured for one or more specific contexts and situations, for example including those that represent the most likely intentions, patterns, behaviors or movements of a PUM in a SEE. In this manner the communications between the agent hosted in a computing environment, for example a sensor, and one or more digital twins that include one or more models for that agent, can be undertaken with tokens.

The resources for an agent can be made available dynamically, for example if an agent is at or approaching the limits of its capabilities, for example as monitored by a second agent or by one or more agent monitoring systems, that first agent can be provided with additional resources, for example memory or processing or may be complimented by further agents that operate on the same data sets, where those further agents have additional capabilities.

Agent operations, including actions, such as for example communications, configurations, calibrations, specifications, and the like, can include voice, text, video, haptic embodiments, or the like. These agents can communicate or interact with one or more stakeholder, including the PUM, to for example, provide advice on a range of events, alerts, patterns or behaviors, such as, actions of, for and to a PUM or other stakeholders, current or future behaviors, interactions with other stakeholders, including for example scheduling or tasks, intentions, both current or future safety, health or wellness awareness, for example in the form of guidance as to the one or more risks that could occur for a pattern, behavior, event, action, interaction or the like.

In some embodiments one or more agent frameworks may be employed, where for example, a device, which can include one or more sensors, can be integrated with a SEE, through manual or automatic processes. Part of this integration can be the installation, through reference or embedding of an agent framework, that can support the operations, locally or remotely of one or more AI/ML agents. This can include such a device providing data sets to one or more AI/ML systems or communicating with such systems, for example to provide data to a stakeholder.

For example, when monitoring breathing rate of a PUM, potentially including oxygen consumption, a camera can be deployed which is pointed at chest and the data sets therefrom can be processed by a pipeline that calculates breathing rate or other breathing characteristics, for example depth of breath, length of breath and the like. In this example, the camera can be moved or refocused to the nose of the PUM to identify any nasal breathing difficulties, which can include an audio-processing pipeline, for example one embodied in hardware, such as an ASIC, SOC or specialist audio hardware, which can then calculate or estimate breathing rate.

For example, a camera can be physically or logically directed to different focus points, for example chest, nose, to generate data sets for processing by one or more pipelines employing one or more algorithms. In some embodiment, different sensors can be arranged with differing configurations for evaluation of breathing (for example mouth open/closed), which can be active at differing times of day and in different situations, for example when the PUM is sleeping, exercising, resting reading and the like. This can involve differing hardware or sensors, devices or systems configurations.

These data sets can inform one more systems, including care processing or care hubs to initiate one or more action, for example to adjust directly or through instruction to another stakeholder, such as a carer, physical devices, such as oxygen masks, beds, chairs or other furniture, including those that have remote operations, or increase oxygen flow, change HVAC settings and the like.

### Agent Deployments

In some embodiments, one or more monitoring system, including AI/ML systems can undertake the evaluation of the state of the SEE and determine, the deployment or configuration of one or more agents in response to that evaluation.

For example, a minimal set of agents may be deployed when the state of the SEE is determined to be quiescent, and as that state changes, further agents may be deployed or configured in response to those changes.

These deployments can include various types of agents, as described herein, which are configured for the conditions of the SEE as measured by the one or more sensors, devices or systems therein.

In some embodiments there can be an agent manager system which undertakes the configuration or deployment of one or more agents, for example those in an agent framework.

In some embodiments, an agent framework can comprise sets of agent specifications or agent embodiments that have not yet been fully configured or deployed. This can include those agent types described herein and may include agent frameworks in such languages as python, for example Atomic agents, LangChain and the like.

In some embodiments, this can include one or more pre-positioned, preconfigured or pre-deployed agents that can be initialized for minimal reaction timing to a change of state including an event, action, occurrence, stakeholder instruction and the like. This can include the actions, events, occurrences, including verbal and non-verbal communications of one or more stakeholder, including the PUM.

In some embodiments, agents can be deployed and activated by one or more systems, including for example care processing or care hubs or one or more specifications from an authorized and authenticated entity, such as a stakeholder, for example carer. Once deployed, activated and operating, the agents may be deactivated upon certain conditions, specifications, activities, events and the like. For example, an agent may have completed a task or the current state of the environment or PUM no longer meets the criteria for that agent deployment. In this example, the data the agent has acquired during the operations can be stored in one or more repository.

In this manner these data sets can be made available to multiple instances of that agent or agent deployment as part of a configuration. These data sets can also be used as training data for one or more AI/ML system, including agents. For example, such data sets can include the persisted incoming data set, for example a system prompt or specification, which can be used by other agents, including multiple instances of the same agent deployed at different times. This can also include one or more persisted models, including parts thereof, that can be used, in whole or in part by the one or more agents. These data sets, their use and the operations of the agents, can be persisted and used as further training data for one or more AI/ML system, including LLMs or agents.

In some embodiments, one or more agent frameworks can comprise a set of agents that are preloaded with specific configurations, potentially for specific situations that can occur, for example those predicted by an AI/ML system for example using one or more digital twins. For example, if a fall or other health, wellness or safety event is predicted as likely occur, one or more agents may be configured and deployed to communicate with the PUM or other stakeholders. This can include making the PUM aware of this prediction and notifying them through visual, audio or haptic means that mitigating action should be undertaken. This can include configuration of one of the devices capable of communicating with the PUM, including smart TV. Smart phones, other worn or carried devices, smart speakers and the like. This can also include communicating with one or more carer who may then intervene with the PUM to mitigate the potential event, for example by having them sit or lie down or otherwise providing physical support. In some circumstances automated systems such as robotic cleaners, HVAC or other environment systems, appliances, such as stoves and the like may be configured to an operational state that reduces any risk to the PUM, for example they may be turned off.

In some embodiments the identification of a pattern or behavior of a PUM, including sequences thereof, can result in the deployment of one or more agent. This can include deployment and resource management and optimization of those agents, so that the monitoring of the PUM is undertaken in a manner that optimizes the available resources, such as energy supply, memory, processing and the like. This can include changing the operating conditions of one or more agents from, for example, active monitoring and subsequent data generation to passive operations, where the agent monitors one or more sensors but does not generate any data sets unless and until there is, for example, a threshold event or the agent can be configured to a passive state, where until a specific communication is received, the agent will consume minimal resources.

In some embodiments, agents may be tokenized in that they from part of the payload of a token, for storage, distribution or deployment. These tokenized agents may be positioned in sensors, devices or systems in preparation for one or more events. For example, if an event is detected by a sensor, device or system that is present in a SEE, for example a health, wellness or safety event impacting a PUM, then a trigger condition can be communicated to the one or more tokenized agents, that result in those agents being instantiated, configured and becoming operating in response to that trigger.

In some embodiments, one or more agent by be deployed based on a number of criteria or techniques, such as when one or more trigger or threshold is breached or a trend that indicates such breach is recognized, one or more agents can be deployed and configured to respond. These triggers or thresholds can include the measurement of the state of an environment or the PUM therein by the one or more sensors, devices or systems present in a SEE.

In some embodiments one or more agent can be deployed and configured to monitor one or more sensors, devices or systems present in a SEE and on detection, recognition or other matching data, can invoke the deployment of one or more further agents in response. For example, if a certain pattern or sets thereof, including sequences, matches the configuration of a first agent, then that agent can invoke the deployment and configuration of a second agent. This can include the measurement of the state of the environment or the PUM therein, in whole or in part.

In some embodiments, the data sets generated during a period, at a location or in response to or in advance of one or more event, can be retained in one or more repository. This can include any processing of these data sets, including the systems of that processing, any methods employed and any outcomes there from. In this manner this retention of data can be between deployments.

In some embodiments, a sensor, device or system may be recognized through one or more communications capabilities, for example, WIFI, Bluetooth and the like, which can include the use of tokens for these communications, where based on recognition, an agent framework can be made available such that when deployed, the sensor, device or system capabilities may be evaluated, in context of a SEE, such that for example, the sensor, device or system capabilities may extend or expand SEE capabilities or operations. For example, when a sensor, device or system is present in the SEE, these capabilities may be accessed, with the appropriate permissions, authentication or authorization and can include one or more AI/ML agent frameworks, including local or remote processing, including any AI/ML operations.

In some embodiments, an agent framework may be configured, for example using a PUM HCP or specific PUM data sets, patterns or behaviors that personalize that agent to a PUM or other stakeholder. This can include agents that are configured or deployed that become activated and operational when certain state changes, for example, an event, threshold, location or other trigger. This can include the use of one or more repository, for example including one or more LLM, which forms a knowledge base for that PUM, stakeholder, SEE, PPE, location or specific entity. In this manner one or more sensor, device or system can employ one or more agents, through reference or embedding, such that these agents can be dynamically generated or deployed, including in response to one or more thresholds, state changes, alerts, events, patterns or behaviors, including for example those predicted by one or more AI/ML system, including LLMs.

In some embodiments an agent framework or an instantiated agent can include one or more decoder-based transformers for an LLM.

In some embodiments, differing types of agents configured for different contexts, situations or objectives can be instantiated, using for example, an agent framework and one or more configuration specifications. This can include specifications that have been generated from or in response to one or more PUM activities, including patterns or behaviors. For example, if PUM behaviors, as measured by the one or more sensors, devices or systems of the SEE, indicate a change in the condition of the PUM, for example increasing breathing difficulties, then one or more agents can be deployed to monitor that condition, research further data on that condition, manage and inform one or more other stakeholders, monitor for conditions that are likely to occur based in that condition, undertake one or more actions, for example, configure or manage one or more sensors to improve the monitoring of that condition or the like.

In some embodiments, this can include the instantiation, deployment, configuration or operations of one or more AI based agents including those described herein.

FIG. 10 illustrates agent deployment or configuration, according to an example embodiment of the present disclosure. A PUM (1004) may be domiciled or present in a sensor-enabled environment (SEE) (1001). The SEE can include one or more sensors (1002) which can be bound to or have a relationship with one or more agents (1003). The SEE can also have one or more stakeholders (1005) present and may include one or more agent IoT devices (1006). The deployment or configuration of the one or more agents in the SEE can be undertaken by agents that facilitate use of a device, sensor or system (1007). The agents can be deployed by an agent deployment system (1008), which can use one or more agent frameworks (1009). The deployment or configuration can be initiated, for example, by a monitoring system which can include one or more patterns (1010) or behaviors (1011) which can determine, at least in part the configuration or deployment of the one or more agents.

### Research Agents

A research agent can be configured to seek data sets that are specific to a particular topic, condition, situation, occurrence, or authorized instruction. This can include measured conditions of the PUM, for example as provided by data sets generated by the one or more sensors, devices or systems of the SEE.

For example, the PUM can instruct a research agent to provide data about a particular condition, for example heart, breathing, recovery time and the like. The agent can interact with multiple data sources, including LLMs and AI systems, such as Claude, Chat GTP, Perplexity or other data sources such as NIH and the like.

The configuration of the research agent can include goal or objective setting and may include specifications that include, condition(s) of PUM, selection of data sources and may be configured to identify likely health/safety conditions.

### Discovery Agents

A discovery agent can be configured to identify data sources for one or more PUM or other stakeholder. This can include querying one or more sensors, devices or systems for data sets that match the configuration of the agent. The discovery agent may use, for example, other AI/ML systems, such as LLMs. They may also access MCP servers, to query such sources for data sets that match the agent configuration. The agent may interact with these other systems through, for example, a prompt or other interface.

In some embodiments discovery agents can be configured to undertake the gathering or extraction of data that matches the configured criteria. The agent can be configured to analyze that data and can act on results of that analysis through communications with other agents, sensors, devices or systems. This can include the identification of state of the environment or PUM

### Execution Agents

In some embodiments an execution agent can be configured to undertake one or more specific actions in response to a set of conditions, an instruction from care processing or other monitoring systems, event, action or occurrence or a change in the context or state of a PUM.

Execution agents can include task directed agents that can be configured to undertake a task. The intended outcome of a task is matched to a specified objective within a frame of reference. For example, this can include the configuration of one or more sensors, devices or systems through for example, increasing fidelity, applying different filters, invoking other sensors for differing spectra and the like.

For example, an execution agent may invoke other AI/ML systems. This invocation may occur, for example, if a trigger, event or alert is identified by the execution agent. For example, the agent might invoke an LLM for transcription/audio identification, which can process a voice communication with a PUM or other stakeholder. In this example the execution agent can be configured to invoke or configure other sensors, determine which alerts or actions to undertake, for example, alerting a carer or invoke one or more other agents.

### Specialist Agents

In some embodiments, specialized agents combining traditional LLMs and other ML/AI techniques with motion LLMs or tools, may run complex multi-agent processes. Capabilities of these agents may include pattern or behavior detection, prediction, response, action planning or execution. Based on these actions, such agents may generate notifications to stakeholders or other communications to one or more sensors, devices or systems, e.g., those in a SEE. This can include, for example, agents that are health condition-focused, such as heart, breathing or other body sub systems, such as mobility, eyesight, hearing and the like. Such agents may identify a PUM physical state, such as if they have fallen, are sitting or lying down.

### Recovery Agents

In some embodiments an agent may be configured with specifications of a predicted recovery for a PUM from a procedure. For example, hip, shoulder, knee or other surgery can have recovery milestones for the PUM. In this example the recovery agent can be configured with these specifications and can configure the one or more sensors, devices or systems present in a SEE, including for example sensors that have been installed for this purpose, to measure or evaluate the PUM's recovery. This can include configuration of the one or more sensors, devices or systems to increase the efficiency, fidelity, quality, frequency or other configurations of the one or more sensors, devices or systems.

### Response Agents

In some embodiments an agent may be configured to undertake one or more specific responses to a situation to which the agent is aware. For example, there may be a set of response agents each configured to respond to a specific state, event, alert, occurrence, activity or pattern or behavior of a PUM or other stakeholder. For example, a response agent can be instantiated and made operational by a stakeholder, including the PUM or carer. This can include sets of response agents which can be configured, deployed and operated in a sequence. These response agents may share data sets with other response or other agent types to ensure continuity of operations and retention of data where appropriate.

It will be appreciated there may be further agent types that are instantiated dynamically with a set features and resources, and that provide other capabilities.

### Intentions, Behaviors and Patterns

Components of the SEE may be configured for the evaluation of the potential intentions of a PUM based, at least in part, on the behaviors including patterns, that have been observed or measured by the one or more sensors, devices or systems of the SEE. For example, an AI/ML system, including one or more agents may use one or more digital twins to predict one or more behaviors, including sequences thereof, based, at least in part, on an initial data set that, for example, includes movement detection or emulation involving one or more PPE, of a PUM.

In some embodiments a PUM who is present in a SEE may have an intention to undertake a behavior, task, action or other activity, some of which can include the PUM's movement. The PUM can have other intentions, such as watching a particular TV show, reading a book, drinking a beverage and the like, which can also include some movement elements.

In these cases, the SEE can employ a PPE to evaluate these movements to, at least in part, determine the intended possible behaviors, actions, activities or other movement-based intentions of the PUM. This can include the use of one or more AI/ML for example using embeddings and vector spaces to, at least in part, determine which movement sequences match the intentions of the PUM. This can include the prediction of the intention or the possible outcomes of such intention, ranked for example by severity or likelihood. For example, an intention may include a physical activity which has a risk metric and the undertaking of that activity, for example running down stairs, can have an increased risk, including of a severe fall. This predicted outcome can be used to generate a notification or alert to a PUM or other stakeholder, for example a carer, that configures one or more device to provide an alert to the PUM or other stakeholder.

In some situations, a PUM initial movements can correspond to a number of possible intentions, such that the AI/ML system or agent may configure a set of digital twins with such initial movement data sets, and using one or more predictive models, to predict the potential or actual intentions of the PUM. This can include comparison of the digital twins with the evolving further movements of the PUM to narrow the possible behaviors the PUM may undertake. Such an approach can yield behavior predictions that can be used to guide, influence, alert or intervene with the PUM so as to assist their intentions, mitigate or avoid risks of those intentions, mitigate or limit health, wellness or safety impacts of those intentions and the consequent behaviors through provision of alerts and notifications to the PUM, a carer or other stakeholder. This can include for example activating a worn or carried device to generate an audio, visual or haptic output.

In some embodiments, a set of digital twins can have an initial movement, with differing variations, based at least in part on the patterns or behaviors of the PUM, where one or more further AI/ML can operate to evaluate the digital twins to correlate the most likely pattern or behavior of the PUM or other stakeholder with the data sets generated by the one or more sensors, devices or systems of the SEE involved in monitoring the PUM or other stakeholders. This determination can result in an output of the AI/ML to configure one or more of the SEE sensors, devices or systems or to notify or communicate with one or more stakeholders, including the PUM through their one or more devices that are controlled by them.

For example, one or more AI/ML systems, including one or more agents, that form part of a SEE may provide, for example assistance to the PUM. For example, this may include n operating doors, windows or other automated systems, such as HVAC, lighting, kitchen appliances and the like. This may also include, providing the PUM with instructions, recommendations, specifications or other communications including controls or other data sets that may aid them in undertaking a particular intention. For example, if there is a specific medicine that the PUM needs to take at a particular time, the systems may provide advance notifications of that forthcoming event, through configuration or activation of a worn or carried device to generate an audio, visual or haptic output. This notification can include text, visual, audio, haptic or other forms of human interpretable communications and may also involve interaction with one or more IoT devices or systems, such as automated medicine dispensers and the like.

The SEE may also provide the PUM with assistance to improve their safety. For example if a particular medicine lowers blood pressure or has a known side effect, the system may prompt the PUM to sit or lie down for a period after consuming the medicine. In another example, the system may prompt the user to eat something prior to taking a medicine that requires them to do so. This can be managed through the patterns or behaviors of the PUM and the AI/ML systems models of those behaviors.

In some embodiments where one or more intentions have been predicted, using for example one or more digital twins, one or more agents can be configured or deployed in anticipation of that intention resulting in an action by a PUM. For example, if a PUM has a set of behaviors that occur at a regular and repeated time, an agent can be configured and deployed in advance of that time to activate, operate, or configure the environment or devices therein to support the PUM in the successful implementation of those intentions. For example, this can include evaluation of the risk or safety metrics.

In some embodiments, one or more AI/ML system, including one or more agent can be employed to monitor a PUM intention, and as the that intention unfolds into an action or activity, can be configured to observe or measure that activity. This can include the use of one or more digital twins to generate that activity in the digital twin, using for example a PPE, and for the any difference between the measured and observed PUM activity and the digital twin activity to be measured by the AI/ML system or agent to, at least in part, determine any functional deterioration in the PUM execution of the activity. This can include providing the PUM with data as to how to reduce any one or more risks of the activity, improve their performance of the activity, through configuration or operation of one or more devices or systems present in the SEE, for example using audio, text, visual, haptic communications and the like. For example, such devices can include smart TV's, smart phones, smart watches or any other capable device. In some circumstances these communications can be communicated to one or more other stakeholder, such as a carer, who may then assist the PUM.

### Responses and Interactions

Interactions between the SEE and one or more stakeholder, including the PUM can include voice, text, visual, haptic or other human or machine-readable communications. This can include, for example, icons or graphics that are representations of actual or potential situations, such as danger, caution, instructions to a PUM to stop, start or pause a particular activity and the like. For example, the SEE monitoring or management systems may use one or more applications or agents to interact with the stakeholder. For example, when communicating with the PUM this may be through one or more devices capable of providing the relevant communications capabilities, such as a PERS, smart phone, smart watch, smart speaker, smart TV or the like or specialist hardware such as an earpiece, for example one that is used as a hearing aid.

Where the stakeholder is not human, for example an insurance company, health provider, care scheduling system or the like, then the data sets from or to the SEE management systems may be formatted for such communications, using for example, an API or an MCP server.

In some embodiments, there can be one or more response system that is configured to accept communications from one or more other systems, including the one or more sensors, devices or systems of a SEE, in various formats, such as data sets, patterns, behaviors, state data, tokens and the like. These response systems can then initiate communication to one or more other entities including for example, stakeholders, including the PUM.

In some embodiments, a care hub, care processing or monitoring systems may include a response system, which can communicate with one or more other systems elements candidate responses to current or predicted states, events or actions of a PUM or other stakeholders in a SEE. This can include response candidates that can calibrate or configure the one or more sensors, devices or systems that are deployed or present in a SEE. These response candidates may include data sets, patterns, behaviors, state, configurations, calibrations, one or more language expressions, for example pattern or behavior, or other elements in any arrangement.

In some embodiments, this can include the use of one or more tokenized language expressions, for example a pattern or behavior language, such as a set of tokens, for example a set of X tokens comprising token A/B/C which equates to a current or predicted monitoring situation

In some embodiments one or more AI/ML systems, including agents, may contribute to determining one or more response candidates that include characteristics, including metrics of one or more languages representing the state of the PUM in a SEE. In this manner such systems may operate to evaluate the potential of the response candidates to impact the quality of life of a PUM.

For example, AI/ML systems, including agents, employing techniques such as Retrieval Augmented Generation (RAG) and/or context sources such as MCP servers may be employed to, at least in part determine potential response candidates, which can include the use of digital twins, physics engines, with which to assess or evaluate one or more contextual behaviors of a PUM in a SEE.

In some embodiments there can be one or more response frameworks, which represent particular responses to likely, well known or predicted events affecting a PUM. For example, there can be one or more fall response frameworks, which can be instantiated depending on the severity of the fall as detected by the one or more sensors, devices or systems of a SEE, which for example may be represented by one or more pattern or state. These response frameworks may be stored in one or more repository and may be made available to multiple PUM domiciled in multiple SEE. For example, there may be a response candidate that is configured for immediate response, such as calling an emergency response telephone number (911 in the U.S.) or other emergency services. There also may be one or more standardized response frameworks, including standardized responses for known PUM adverse situations, such as falls and the like.

In some embodiments, response frameworks may comprise one or more data sets generated by one or more sensors, devices or systems deployed or present in a SEE, patterns of such data sets, state data, one or more categorized data sets, including spatial, temporal, contextual behavior or wellness or other data, including calibration or configuration data in any arrangement.

The use of monitoring to support a PUM in a sensor-enabled environment (SEE) can include the provision of one or more risk assessment systems coupled with one or more response systems. In some embodiments these capabilities may be integrated to provide a monitored environment that can, in whole or in part, evaluate the health, wellness or safety risk(s) for one or more PUM through their habitation and activities within that environment. This risk evaluation data can inform one or more response systems, which may then communicate these risk evaluations to one or more other systems or stakeholders, including for example, emergency response teams, to avoid or mitigate these risks. Inherent in any response system is the potential or actual impact of those responses for or on the PUM and as such in some embodiments one or more impact assessment systems may operate in collaboration, for example, as part of a dynamic feedback mechanism to further consider, evaluate, model or predict these impacts, which can in turn lead to those responses being modified or varied so as to effect the most beneficial outcome for a PUM in the prevailing circumstances.

In some embodiments, one or more pattern frameworks, comprising at least in part, data sets or patterns generated by the one or more sensors, devices or systems of a SEE can be correlated with event pattern frameworks, representing the potential health and wellness event that can impact a PUM. Such an alignment may be stored in one or more repository, where one or more AI/ML system, including agents, can using for example, one or more digital twin or one or more physics engine, determine, at least in part the likely correlations of the pattern frameworks and event frameworks as each of these is populated by the data sets or patterns generated by the one or more sensors, devices or systems of the SEE. This determination can include the use of game theory, where for example, one or more strategies are employed or identified to establish correlation or causation. These determinations may, in some embodiments be stored in one or more repository.

In some embodiments, determinations can form one or more response frameworks, where upon population of the one or more pattern frameworks or event frameworks with the data sets or pattern generated by the one or more sensors, devices or systems of a SEE, one or more thresholds, metrics or other variables may be satisfied, such that the care hub or care processing systems may initiate one or more response candidate based, at least in part on the one or more response frameworks.

In some embodiments, one or more care hub or care processing systems may include the use of QoL (Quality of Life) metrics, which can include impact analysis, for the selection or deployment of one or more response candidates.

FIG. 11 illustrates a monitoring and response environment integrated with a SEE, according to an example embodiment of the present disclosure. The SEE includes one or more sensors, devices or systems (1102, including those worn or carried by a PUM (1104), one or more agents that have a relationship with those sensors (1103), one or more stakeholders (1105) and one or more agent IoT devices (1106). One or more monitoring systems (1107), for example care hubs or care processing can be employed to identify patterns (1108) or behaviors (1109) of the PUM (1104) or stakeholders (1105) within the SEE (1101). These monitoring systems (1107) can then communicate one or more responses or response candidates to one or more response systems (1110). The response systems can include risk systems (1111) or agents (1112). The response systems can communicate such responses to the SEE (1101) or to the entities therein, including the PUM (1104), e.g., by sending messages to their one or more worn or carried devices, including for example those under their control such as a smart phone, to one or more stakeholders (1105), one or more sensors, devices or systems (1102) and any agents thereof (1103) or one or more IoT agent devices (1106). In some embodiments agents (1112) controlled or managed by the response systems may communicate with such IoT devices (1106).

Many of the interactions with the PUM that are conducted by one or more AI/ML systems, including agents, forming part of the SEE can be configured to reflect the PUM's communications preferences and choices. For example, if the PUM prefers to receive communications through a single device or through a single mode, for example text, then then components of the SEE interacting with the PUMP can be configured to reflect these preferences. In addition, the one or more AI/ML systems may observe communications with the PUM and support their efficacy by comparing a PUM's intentions to their actions. In this manner the AI/ML system may modify such communications to assist the PUM in successfully achieving their intentions. In some cases this may occur without the PUM expressly stating or communicating such intentions. The AI/ML system monitoring the behaviors of the PUM can determine the possible behaviors the PUM intends and within the capabilities of the AI/ML systems and the SEE assist the PUM in achieving a successful outcome for their intentions.

Interaction timing can be crucial to the acceptance of the communications content by a PUM, for example, if a message is continuously communicated there can be a tendency to for a PUM or other stakeholder to ignore or devalue the content, which can create a detrimental impact. In some embodiments, communications and actions resulting therefrom can be used to generate a set of metrics for evaluation of the efficacy of those communications to the one or more stakeholders through, at least in part, data sets, patterns or behaviors that are impacted by the contents of the communications and the responses of the stakeholder(s), including a PUM, thereto.

In some embodiments, one or more AI/ML system, which may include one or more digital twins, may be employed to determine, in part or in whole, the optimum timing for communications to or about a stakeholder, for example a PUM. This can include the use of game theory modules and the games thereof to develop one or more strategies for optimizing the impact of the content of such communications. Such strategies can include, for example the frequency with which a single or multiple communications are provided to a stakeholder, in part to ensure that the stakeholder applies sufficient attention to the content of these communications, such that they act or react to them in a manner that is beneficial or mitigates any detrimental outcomes. Determining the correct strategy can include such systems using, for example, AI/ML models of, for example, the intentions of a stakeholder, including their observed or predicted behaviors, where communications may be conveyed to a stakeholder informing them or advising them of one or more risks for those intentions or behaviors. In some embodiments an agent, for example one operating on a worn or carried device may include configuration options to facilitate the stakeholder to vary the number and type of communications. In some versions thereof, such options can be constrained by one or more other stakeholders, such as family or a carer.

One aspect of this approach is the use of various timing mechanisms, such as clock time, behavior time, personalized time or other temporal metrics to optimize the understanding, acceptance or actions based on these communications. For example, if a PUM is a coffee drinker, then presenting communications in advance of their initial, morning coffee may not be an optimum timing, whereas after that initial coffee, the PUM's alertness to the communications may have increased.

In some embodiments, a watchdog process may operate on one or more data sets, patterns or behaviors to detect, in whole or in part, their state, including whether that state is quiescent or not. This can include one or more AI/ML agents configured to deploy and operate as watchdogs.

In some embodiments, a game theory system may operate as a watchdog to ensure that that AI/ML systems can only operate to achieve the predetermined goals of the game. For example, in controlling an HVAC system, there may be a range of setting which match the goals of the game, for example in a heat stress condition, only cooling satisfies the game parameters.

In some embodiments a PUM or other stakeholder relationship with the SEE, including the one or more sensors, devices or systems, including one monitoring systems, such as care hubs and care processing systems and any one or more AI/ML systems thereof, including LLMs which can be represented by one or more agents active on the devices of the PUM or other stakeholders and can include various degrees of trust.

These trust relationships may be at the granularity of an individual sensor, for example a camera. However, the data sets generated by such a sensor, may be tokenized and processed by, for example, a monitoring system, which can include one or more AI/ML systems. The output of that processing, for example a communication to a PUM or stakeholder regarding a risk, metric or other communication or interaction may be presented to the PUM or other stakeholder through, for example an agent embedded on a device, The accuracy, timeliness, validity and veracity of that such communications can, in part or in whole influence the levels of trust in the system, or a particular sensor, or type of communication.

For example, if a communication continually provides a risk metric, for example a warning, every time the PUM undertakes a specific behavior, for example taking a shower, the PUM is less likely to trust any communications that may represent a clear and present danger to their well-being, health or safety.

In some embodiments, a SEE can include agents or other devices or systems, which can incorporate one or more AI/ML system. These included agents or other devices or systems may provide interactive PUM engagements. For example, a communication may be an inquiry to the PUM, such as “what should you have/would you like for lunch?”. These interactions can provide a set of communications that can assist the PUM or other stakeholder in avoiding potential risks or enhancing their care, wellness, health and safety through proactive behavior reinforcement. For example, such communications or interactions can include, reminders, prompts, events, actions, calendar, appointments, meetings, scheduling, suggestions, cautions including safety awareness, risk metrics, sensor, device or systems configurations or the like.

In some embodiments, as the personalization of the monitoring systems, including the AI/ML systems, evolves to more closely match the particular characteristics of the PUM and any stakeholders involved with them. When personalized, these monitoring systems may become more trusted by the PUM or other stakeholders. For example, as the monitoring systems undertake the measurements and predictions of current or future behaviors, the relationship of the PUM or other stakeholders is, at least in part, based on the accuracy of any predictions, the utility or benefit of any communications and perceived value of any responses.

In some embodiments, one or more agents that are capable of interacting with one or more stakeholder, including the PUM, can for example, provide multiple potential responses, communications or predictions. The chosen communications may present outcomes for a PUM that, to a greater or lesser degree, provide some benefits to the PUM. In this situation, such an agent may operate to collaborate with the PUM on the selections, decisions or responses in an interactive manner. The PUM may be offered a set of alternatives or may engage with the agent, through for example a prompt system, employing text, voice, visual, haptic or other communications, such that the selected response is determined in collaboration with the PUM.

In some embodiments, the system may provide similar collaboration capabilities to various stakeholders in place of the PUM. This may occur, for example, when the PUM has significant deterioration of their mental faculties and one or more stakeholders is delegated to assist the PUM in their decision making. Delegation may be partial, e.g., there may be a separation of such delegation, authorities, authorizations, communications or responses. For example, the monitoring systems may include delegated certain levels of authority or decision making, for example providing communications on scheduling, appointments, medication timing or the like. In such case, a stakeholder may retain other authorities or decision making. For example, some authority or decision making can involve multiple parties in part or in whole, including monitoring systems or other agents, PUM or other stakeholders in any arrangement.

Various monitoring systems may, in some cases, operate in collaboration with one or more stakeholders, which can include the PUM. Such systems may be configured and operated to predict, anticipate or support one or more desired behavioral outcomes. For example, if a PUM behavior has a natural or detrimental health, wellness or safety impact, for example one measured by one or more risk metrics, the monitoring systems, in whole or in part, may be configured or operated to present and encourage a behavior that is beneficial to the wellness, health or safety of the PUM. This can involve collaboration with one or more stakeholders, for example using one or more agents.

In some cases, an agent may be configured to control one or more devices, for example, a medication dispenser, kitchen or household appliance, wearable sensor, such as an oxygen sensor or environment automation, such as HVAC, windows, blinds, curtains and the like.

In some cases, an agent can be configured to generate a set of specifications, instructions or code for the operations of one or more automation systems or sub systems thereof.

In some cases, an agent configured for a task may also invoke, configure and deploy other agents, for example research, discovery or execution agents to achieve an objective.

FIG. 12 illustrates a flow diagram of an example method, according to an example embodiment of the present disclosure. In the example method 1200, at 1202 a PUM in an SEE may be monitored with various sensors. The sensors may have agents associated with them to process collected data, or may pass the collected data to be analyzed and summarized elsewhere. Once the data is collected, various results may occur. For example, in 1204 a pattern in the monitoring data may be recognized, e.g., by using an AI/ML system with access to historical data for the PUM, and/or data on related populations of individuals, e.g., with similar conditions. In 1205, a change of state of the PUM may be recognized, for example by an AI/ML system, working either directly on the monitoring data, or based on the analysis of results from further analysis of such data, e.g., the output of the pattern recognition in 1204. Similarly, in 1206, a change of state of the environment may be recognized, for example by an AI/ML system, working either directly on the monitoring data, or based on the analysis of results from further analysis of such data, e. g., the pattern recognition in 1205. In response to the analysis of the data, and/or to the recognition of a state change in the PUM or the environment, action may be taken. To carry out and control the action, an agent may be employed. Based on the data and/or its analysis, In 1208, it may be determined that a new agent of a particular type needs to be configured and deployed, the type depending on the data and its analysis. In 1210 the agent of the new type may be configured and deployed. Alternatively, in 1212, it may be determined that a currently operating agent may need to be reconfigured, e.g., by changing operating parameters of the agent. The agent may be reconfigured in 1214. The new or reconfigured agent may carry out various activities, depending on the analysis of the various data. For example, in 1216 the agent may conduct a conversation with the PUM, either to obtain information or to improve the well being or safety of the PUM. This may be carried out using an appropriate AI/ML system. In 1218, a sensor in the environment may be activated or reconfigured, so that, for example, additional data may be collected in light of the change of state of the PUM or the environment. In 1220 output of a particular sensor may be monitored by the agent, which may later take additional action based on the data obtained by additional monitoring. In 1222, the agent may determine the state of the PUM, or whether state changes have occurred. In 1224 the agent may determine the state of the environment, e.g., who is present in the environment, temperature, lighting, the operating state of various equipment, etc. In 1226, the agent may identify a task that needs to be performed and activate a second agent to carry out that task. Alternatively, an existing operating agent may be reconfigured. In 1228, the agent may identify an intended action of a PUM. IN 1230, the agent may assist the PUM in implementing an intended action, for example by activating various systems, alerting carers, obtaining resources, or providing helpful information. For example, in 1230, using an LLM, the agent may generate a prompt to remind the PUM of information related to their intended action, which in 1232 may be delivered to the PUM, whether through an audio message, a text or email, or via other communications avenues.

It will be appreciated that the various tasks of the agent described above may be carried out different orders, and the results of one task may influence the choice or execution of a subsequent task. It will also be appreciated that multiple agents may be used and the various tasks may be carried out by different agents, possibly with communication or coordination between the different agents.

FIG. 13 illustrates an architecture for an example agent, according to an example embodiment of the present disclosure. The agent 1300 may run locally on a processor 1310, which may be a local hardware processor system including one or more CPUs and/or GPUs. It may also run on a virtual processor, which executes on hardware located remotely in a shared manner accessible via a network, e.g., using a virtualized system running on a cloud service on shared CPUs or GPUs. The agent may have access to storage 1320 accessible to the processor, which may include local memory or disk storage, as well as cloud storage.

The agent may include control logic 1322, e.g., a program running on the processor 1310, which control and coordinates the overall operation of the agent. The storage 1320 may store various information including the control logic 1322, various AI modules such as LLM 1324, and datasets 1326, e.g., data sets received from sensors, or patterns or other information derived from such datasets. The agent 1300 may include or have access to one or more generative AI systems, e.g., LLM 1324, or other types of AI/ML systems. Such as a RAG or MCP. These AI/ML systems may be used to analyze information 1326 received and stored by the agent, to plan for and make decisions within the agents assigned capabilities under direction of the control logic 1322, and to generate communications for the agent to make with other actors, including both humans and other agents, systems, or devices. The agent may have access to historical data stored in a depository 1340, either remotely or locally. The agent may also have access to externally or previously processed data, such as a pattern library or other prior analysis, via a processed data interface 1332. The agent may include or have access to sensor interfaces 1333 which manage communication, control, and data exchange with various external sensors, e.g., sensors in a SEE. The agent may include or have access to a supervisory interface 1334, which allows the agent to launch, communicate, and/or control other agents, or to receive external instructions, e.g., from other agents or systems that control the launch or configuration of the agent 1300. The agent may include or have access to an environmental interface 1336 which provides mechanisms for the agent to control external devices, e.g., IOT devices or other devices in a SEE. The agent may include or have access to a communication interface 1335 that facilitates communication of the agent with various humans through different approaches, including voice or other audio communication, email, text, etc.

Systems, methods, and apparatuses of the present disclosure may be implemented on a variety of devices, such as IPUs, GPUs, DPUs, CPUs, GPUs, ASICs, FPGAs, DSPs, or any other device capable of processing data. Instructions for performing the same may be provided as hardware or firmware on any computer-readable medium including volatile and non-volatile forms of memory. Particular implementations of techniques of the present disclosure may be structured in any number of manners, including a modular program architecture, a monolithic program architecture, on a single device, and distributed across more than one device or processor.

Although certain figures and descriptions have been provided, many additional variations and modifications will be apparent to those of skill in the art. It will be appreciated that presenting all possible variations and modifications is an impractical task, and thus any sequence, particular structural or device implementation, or underlying technique of the present disclosure may be substituted or modified to meet the needs of particular implementations, and that doing so may not depart from the scope of the present disclosure. It will therefore be appreciated that the examples presented herein are presented for illustrative purposes only, and are in no way intended to be limiting of a scope of the present disclosure. It will also be apparent to any individual of skill in the art that various embodiments described herein and elements thereof may be combined as needed to suit any particular implementation, and that doing so does not depart from the scope of the present disclosure. Similarly, terms of “minimize” and “maximize” shall generally be understood to refer to optimizing for a “best” lowest value or highest value, respectively, and may include the identification of local minima, local maxima, a global minimum, or a global maximum, which can vary at different times or under new goals or conditions.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, the term “or” is to be interpreted in the inclusive sense and not the exclusive sense unless explicitly stated otherwise or when clear from the context. Accordingly, recitation of “A or B” is intended to cover the sets of A, B, and A-B, where the sets may include one or multiple instances of a particular member (e.g., A-A, A-A-A, A-A-B, etc.) and any ordering thereof.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a particular member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

As used in the present disclosure, the term “determining” encompasses a variety of actions that may include calculating, computing, processing, deriving, investigating, identifying, looking up (e.g., via a table, database, or other data structure), ascertaining, receiving (e.g., receiving information), accessing (e.g., accessing data in a memory), retrieving, resolving, selecting, choosing, establishing, and the like.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S. C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

The use of “including”, “includes”, “comprises”, or “comprising” should be understood as “including but not limited to”, unless express language indicates that the language should be read as exclusive, e.g., by the use of “consisting”.

1. A method of operating a sensor-enabled care environment, comprising:
monitoring a person under monitoring (PUM) in a sensor-enabled environment (SEE) with plurality of sensors that produce monitoring data; and
based on the monitoring data, automatically deploying or reconfiguring an agent, wherein the deployed or reconfigured agent is configured to carry out at least one action a one selected from the group consisting of:
(a) provide an interactive engagement with the PUM,
(b) prompt the PUM to reduce risk to the PUM,
(c) identify an intended action of the PUM,
(d) assist the PUM in implementing the PUM's intended action,
(e) activate a sensor in the SEE,
(f) reconfigure a sensor in the SEE,
(g) monitor the output of a sensor in the SEE,
(h) determine a state of the environment,
(i) determine a state of the PUM,
(j) activate a second agent, and
(k) reconfigure a second agent.

2. The method of claim 1, further comprising:
based on the monitoring data, determining a state of the PUM has changed to a new state;
based on the new state, selecting a type of agent to be deployed; and
launching the agent having the type selected.

3. The method of claim 1, further comprising:
based on the monitoring data, determining a state of the PUM has changed to a new state;
based on the new state, determining a new configuration of the agent that is to be reconfigured; and
reconfiguring the agent to have the new configuration.

4. The method of claim 1, further comprising:
recognizing a pattern in the monitoring data;
based on the pattern that is recognized, selecting the type of agent to be deployed; and
deploying the agent having the selected type.

5. The method of claim 1, further comprising:
recognizing a pattern in the monitoring data; and
based on the pattern that is recognized, determining the new configuration of the agent that is to be reconfigured;
reconfiguring the agent to have the new configuration.

6. The method of claim 1, further comprising:
conducting, by the launched or reconfigured agent using an AI/ML system, a conversation with the PUM.

7. The method of claim 1, the method further comprising:
identifying an intended action for the PUM, by the launched or reconfigured agent, by at least one of
(a) analyzing historical behavior data for the PUM with an AI/ML system to identify the intended action of the PUM,
(b) analyzing historical behavior data for a population of similar individuals with an AI/ML system to identify the intended action of the PUM,
(c) analyzing data patterns derived from the monitoring data to identify the intended action of the PUM, and
(d) analyzing a model of the PUM as a physical object to identify the intended action of the PUM.

8. The method of claim 7, further comprising:
the launched or reconfigured agent generating, with an AI/ML system, a prompt based on the intended action of the PUM; and
the launched or reconfigured agent, prompting the PUM with the prompt to reduce risk to the PUM.

9. The method of claim 1, the method further comprising:
assisting, by the launched or reconfigured agent, the PUM in implementing the PUM's intended action by at least one of
(a) notifying, by deployed or reconfigured agent, a caregiver to provide assistance,
(b) changing the state of the environment by the deployed or reconfigured agent by activating or changing the state of a device in the environment, and
(c) providing, by the deployed or reconfigured agent, information to the PUM.

10. The method of claim 1, the method further comprising:
activating, by the launched or reconfigured agent, a sensor in the SEE or reconfiguring a sensor in the SEE.

11. (canceled)

12. The method of claim 1, further comprising:
determining, by the deployed or reconfigured agent, a state of the PUM.

13. The method of claim 1, further comprising:
determining, by the deployed or reconfigured agent, a state of the environment.

14. The method of claim 1, further comprising:
activating, by the deployed or reconfigured agent, a second agent.

15. The method of claim 1, wherein the agents are AI/ML agents.

16. An article of manufacture comprising a non-transient computer readable medium having stored thereon instructions configured, when executed by a processor, to cause the processor to carry out the method of claim 1.

17. A system, comprising,
a plurality of sensors configured to monitor a PUM in a SEE and produce monitoring data;
a data repository configured to store historical data regarding the PUM;
a processor in communication with the plurality of sensors and the data repository and configured, based on the monitoring data, to deploy or reconfigure a plurality of agents each of the plurality of agents configured to at least one action selected from the group consisting of:
(a) provide an interactive engagement with the PUM,
(b) prompt the PUM to reduce risk to the PUM,
(c) identify an intended action of the PUM,
(d) assist the PUM in implementing the PUM's intended action,
(e) activate a sensor in the SEE,
(f) reconfigure a sensor in the SEE,
(g) monitor the output of a sensor in the SEE,
(h) determine a state of the environment,
(i) determine a state of the PUM, and
(j) reconfigure or activate a second agent.

18. The system of claim 17, further comprising:
storage accessible to the processor, storing at least one of the monitoring data or patterns derived from the monitoring data, the deployed agent having access to the monitoring data or patterns.

19. The system of claim 17, wherein the agents each further comprise a respective AI/ML model.

20. The system of claim 18, further comprising an AI/ML system, configured to identify patterns in the monitoring data, the processor configured to select the type of agent to deploy or reconfigure based on the identified patterns.

21. The system of claim 18, further comprising an AI/ML system configured to identify patterns in the monitoring data, the agent's respective AI/ML models configured to receive the identified patterns and carry out the action based on the identified patterns.
